(19) United States
(12) Patent Application Publication
(10) Pub. No.: US 2009/0163474 A1
(43) Pub. Date: Jun. 25, 2009

(54) FXR Agonists for the Treatment of Nonalcoholic Fatty Liver and Cholesterol Gallstone Diseases

(75) Inventors: Songwen Zhang, Lansdale, PA (US); Douglas Harnish, Pennsburg, PA (US); Mark J. Evans, Radnor, PA (US); Juan Wang, Wynnewood, PA (US)

Correspondence Address: WYETH/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/253,010

(22) Filed: Oct. 16, 2008

Related U.S. Application Data
(60) Provisional application No. 60/960,925, filed on Oct. 19, 2007.

(51) Int. Cl. A61K 31/55; A61K 31/405

(52) U.S. Cl. 514/213.01; 514/415

(57) ABSTRACT

Provided are certain methods of treating nonalcoholic fatty liver disease with farnesoid X receptor agonists. Also provided are certain methods of modulating levels of keratinocyte-derived chemokine (KC), alanine aminotransferase (ALT), aspartate aminotransferase (AST), cytokeratin 18 (CK-18), matrix metalloproteinase-9 (MMP-9), matrix metalloproteinase-14 (MMP-14), tissue inhibitor of metalloproteinase 1 (TIMP-1), and Cytochrome P450 2E1 (CYP2E1); certain methods of identifying FXR modulators; and certain methods of treating patients with existing cholesterol gallstone disease.

This application claims the benefit of priority to U.S. Provisional Application No. 60/960,925, filed Oct. 19, 2007, the entire contents of which are hereby incorporated herein by reference.

Provided are certain methods of treating nonalcoholic fatty liver disease with farnesoid X receptor agonists. Also provided are certain methods of modulating levels of keratinocyte-derived chemokine (KC), alanine aminotransferase (ALT), aspartate aminotransferase (AST), cytokeratin 18 (CK-18), matrix metalloproteinase-9 (MMP-9), matrix metalloproteinase-14 (MMP-14), tissue inhibitor of metalloproteinase 1 (TIMP-1), and Cytochrome P450 2E1 (CYP2E1), certain methods of identifying FXR modulators, and certain methods of treating patients with existing cholesterol gallstone disease.

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin and mineralocorticoid receptors which bind as homodimers to hormone response elements (HREs) organized as inverted repeats (see, e.g., Glass, supra). A second class of receptors, including those activated by retinoic acid, thyroid hormone, vitamin D₃, fatty acids/peroxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al. (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al. (1992) Genes Dev. 6:329-344), coding for RXRα, -β, and -γ, all of which are able to heterodimerize with any of the class II receptors, although there appear to be preferences for distinct RXR subtypes by partner receptors in vivo (see, e.g., Chiba et al. (1997) Mol. Cell. Biol. 17:3013-3020). In the adult liver, RXRα is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al. (2000) Mol. Cell. Biol 20:4436-4444.

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is expressed in the liver, kidney and intestine, among other locations. It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317. The activity of farnesoid X receptor has been implicated in physiological processes including but not limited to triglyceride metabolism, catabolism, transport or absorption, bile acid metabolism, catabolism, transport or absorption, re-absorption or bile pool composition, and cholesterol metabolism, catabolism, transport, absorption or reabsorption.

Nuclear receptor activity, including the farnesoid X receptor activity, has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis and xanthoma, (see, e.g., International Patent Application Publication No. WO 00/57915), hyperlipoproteinemia (see, e.g., International Patent Application Publication No. WO 01/60818), hypertriglyceridemia, lipodystrophy, peripheral occlusive disease, ischemic stroke, hyperglycemia and diabetes mellitus (see, e.g., International Patent Application Publication No. WO 01/82917), disorders related to insulin resistance including the cluster of disease states, conditions or disorders that make up “metabolic syndrome” or “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), obesity, acne (see, e.g., International Patent Application Publication No. WO 00/49992), and cancer, cholestasis, Parkinson's disease and Alzheimer's disease (see, e.g., International Patent Application Publication No. WO 00/17334).

Nonalcoholic fatty liver disease (NAFLD) refers to a wide spectrum of liver diseases characterized by the accumulation of fat in liver cells. The term nonalcoholic is used because nonalcoholic fatty liver disease occurs in individuals who do not consume excessive amounts of alcohol. Approximately 2 to 5% of Americans have nonalcoholic fatty liver disease. Currently, there is no standard medical treatment. General recommendations include exercise, weight loss, diabetes and cholesterol control, and alcohol avoidance. Nonalcoholic fatty liver disease is also associated with increased prevalence of cholesterol gallstone disease. These disorders have, in common, alterations in lipid homeostasis which may contribute to their pathology.

Cholesterol gallstone disease is characterized by cholesterol precipitation in the bile which can lead to the formation of gallstones in the gallbladder. It has affected as many as 10% of Americans. Currently, cholecystectomy, surgical removal of the gallbladder, is an effective treatment but is invasive and contraindicated for some patients. Noninvasive means of treatment include oral administration of bile acids chenodeoxycholic acid (CDCA) and ursodeoxycholic acid (UDCA). CDCA usage has toxic effects including dose-related diarrhea and liver damage. Furthermore, limited efficacy, the need for prolonged use, and high relapse rates for CDCA and UDCA limit their use.

Effective and safe treatments for nonalcoholic fatty liver disease and cholesterol gallstone disease are needed.

Provided are methods of treating nonalcoholic fatty liver disease (NAFLD) in a patient. The methods include administering to the patient a therapeutically effective amount of at least one farnesoid X receptor (FXR) agonist.

Also provided are methods of modulating the level of at least one of keratinocyte-derived chemokine (KC), alanine aminotransferase (ALT), aspartate aminotransferase (AST), cytokeratin 18 CK-18, Col1a2, TGF-β, a-SMA, at least one MMP, at least one positive acute phase protein, TIMP-1, and Cytochrome P450 2E1 (CYP2E1) in a patient. The methods include providing to the patient an effective amount of at least one FXR modulator, to thereby modulate the level of at least one of KC, ALT, AST, CK-18, Col1a2, TGF-β, a-SMA, at least one MMP, at least one positive acute phase protein, TIMP-1, and CYP2E1 in the patient.

Also provided are methods of identifying a FXR modulator. The methods include administering a test agent to a mammal; determining at least one of the following features of the mammal, in the presence of the test agent: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress; and (i) acute phase response in the liver; comparing the at least one feature in the presence of the test agent to the at least one feature in the absence of the test agent; and identifying the test agent as an FXR modulator if the level of the at least one factor is modulated in the presence of the test agent compared to the level of the at least one factor in the absence of the test agent.

Also provided are further methods of identifying a FXR modulator. The methods include administering a test agent to a mammal; determining the level of at least one of the following factors in the mammal, in the presence of the test agent: tumor necrosis factor α (TNFα), monocyte chemotactic protein-1 (MCP-1), KC, ALT, AST, CK-18, Col1a2, TGF-β, a-SMA, at least one MMP, at least one positive acute phase protein, TIMP-1, and CYP2E1; comparing the level of the at least one factor in the presence of the test agent to the level of the at least one factor in the absence of the test agent; and identifying the test agent as a FXR modulator if the level of the at least one factor is modulated in the presence of the test agent compared to the level of the at least one factor in the absence of the test agent.

Also provided are further methods of treating nonalcoholic fatty liver disease in a patient. The methods include administering to the patient a therapeutically effective amount of at least one FXR agonist. In some embodiments, the at least one FXR agonist is identified by administering a test agent to a mammal; determining at least one of the following features of the mammal in the presence of the test agent: (a) neutral lipid deposition, (b) intracellular lipid droplet formation, (c) Kupffer cell activation, (d) inflammatory cell infiltration, (e) inflammatory cholangitis, portal inflammation, (g) fibrosis, (h) oxidative stress; and (i) acute phase response in the liver; comparing the at least one feature in the presence of the test agent to the at least one feature in the absence of the test agent; and identifying the test agent as a FXR agonist if the at least one feature is reduced in the presence of the test agent.

Also provided are further methods of treating nonalcoholic fatty liver disease in a patient. The methods include administering to the patient a therapeutically effective amount of at least one FXR agonist. In some embodiments, the at least one FXR agonist is identified by administering the test agent to a mammal; determining the level of at least one of the following factors in the mammal in the presence of the test agent: vascular cell adhesion molecule-1 (VCAM-1), intercellular adhesion molecule-1 (ICAM-1), TNFα, MCP-1, KC, ALT, AST, CK-18, TIMP-1, Col1a2, TGF-β, a-SMA, at least one MMP, at least one positive acute phase protein, CYP2E1, fatty acid synthase (FAS), small heterodimer partner (SHP), bile salt export pump (BSEP), and multiple drug resistance-2 (MDR2); comparing the level of the at least one factor in the presence of the test agent to the level of the at least one factor in the absence of the test agent; and identifying the test agent as a FXR agonist if it has at least one property selected from reducing the level of at least one of VCAM-1, ICAM-1, TNFα, MCP-1, KC, ALT, AST, CK-18, Col1a2, TGF-β, a-SMA, at least one MMP, at least one positive acute phase protein, and CYP2E1, modulating the level of TIMP-1, and elevating the level of at least one of FAS, SHP, BSEP, and MDR2 in the mammal.

Also provided are methods of treating a patient with existing cholesterol gallstone disease. The methods include administering to the patient a therapeutically effective amount of at least one FXR agonist.

Also provided are methods of treating a patient with existing cholesterol gallstone disease, in which the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress, and acute phase response in the liver, and an elevated level of at least one of VCAM-1, ICAM-1, TNFα, MCP-1, KC, TIMP-1, Col1a2, TGF-β, a-SMA, at least one MMP, at least one positive acute phase protein, CYP2E1, ALT, AST, and CK-18. The methods include administering to the patient a therapeutically effective amount of at least one FXR agonist.

Also provided are methods of identifying a FXR modulator. The methods include administering a test agent to a mammal; determining at least one of the following features in the mammal in the presence of the test agent: (a) gallstone incidence, (b) gallstone dissolution time, (c) bile cholesterol lipids, (d) bile salt/phospholipid ratios, (e) biliary symptoms, and (f) gallbladder inflammation; comparing the at least one feature in the presence of the test agent to the at least one feature in the absence of the test agent; and identifying the test agent as a FXR modulator if the at least one feature is modulated in the presence of the test agent compared to its state in the absence of the test agent.

Also provided are further methods of treating a patient with existing cholesterol gallstone disease. The methods include administering to the patient a therapeutically effective amount of at least one FXR agonist. In some embodiments, the at least one FXR agonist is identified by administering a test agent to a mammal; determining at least one the following features in the mammal in the presence of the test agent: (a) gallstone incidence, (b) gallstone dissolution time, (c) bile cholesterol lipids, (d) bile salt/phospholipid ratios, (e) biliary symptoms, and (f) gallbladder inflammation; comparing the at least one feature in the presence of the test agent to the at least one feature in the absence of the test agent; and identifying the test agent as a FXR agonist if the at least one feature is reduced in the presence of the test agent compared to its state in the absence of the test agent.

Also provided are further methods of treating a patient with existing cholesterol gallstone disease. The methods include administering to the patient a therapeutically effective amount of at least one FXR agonist. In some embodiments, the at least one FXR agonist is identified by incubating a test agent with a cell; determining the level of at least one of the following factors in the presence of the test agent: VCAM-1, ICAM-1, TNFα, MCP-1, KC, ALT, AST, CK-18, CYP2E1, TIMP-1, Col1a2, TGF-β, a-SMA, at least one MMP, at least one positive acute phase protein, FAS, SHP, BSEP, and MDR2; comparing the level of the at least one factor in the presence of the test agent to the level of the at least one factor in the absence of the test agent; and identifying the test agent as a FXR agonist if the test agent has at least one property selected from reducing the level of at least one of VCAM-1, ICAM-1, TNFα, MCP-1, KC, ALT, AST, CK-18, CCYP2E1, Col1a2, TGF-β, a-SMA, at least one MMP, at least one positive acute phase protein, modulating the level of TIMP-1, and elevating the level of at least one of FAS, SHP, BSEP, and MDR2.

Also provided are further methods of treating a patient with existing cholesterol gallstone disease. The methods include administering to the patient a therapeutically effective amount of at least one FXR agonist. In some embodiments, the at least one FXR agonist is identified by administering a test agent to a mammal; determining at least one of the following features of the mammal in the presence of the test agent: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver; comparing the at least one feature in the presence of the test agent to the at least one feature in the absence of the test agent; and identifying the test agent as a FXR agonist if the at least one feature is reduced in the presence of the test agent compared to its state in the absence of the test agent.

### DESCRIPTION OF DRAWINGS

FIG. 1 shows the serum level of alanine aminotransferase (ALT) activity in mice fed a standard chow diet, vehicle treated mice fed a Paigen diet, and FXR agonist, Compound A (isopropyl 3-(3,4-difluorobenzoyl)-1,1-dimethyl-1,2,3,6-tetrahydroazepino[4,5-b]indole-5-carboxylate) treated mice fed a Paigen diet.

FIG. 2 shows the serum level of monocyte chemotactic protein-1 (MCP-1) in vehicle and Compound A treated mice fed a Paigen diet.

FIG. 3A shows the expression level of vascular cell adhesion molecule 1 (VCAM-1), FIG. 3B shows the expression level of intercellular adhesion molecule-1 (ICAM-1), and FIG. 3C shows the expression level of tumor necrosis factor α (TNFα) in the livers of mice fed a standard chow diet, vehicle treated mice fed a Paigen diet, and Compound A treated mice fed a Paigen diet.

FIG. 4 shows representative images of histological sections of livers from mice fed a standard chow diet, vehicle treated mice fed a Paigen diet, and Compound A treated mice fed a Paigen diet. Sections were stained with Oil Red O, hemotoxylin and eosin (H&E), or Trichrome.

FIG. 5 shows images of histological sections of livers from vehicle and Compound A treated mice fed a Paigen diet. Sections were stained with Oil Red O or H&E.

FIG. 6 shows the liver expression level of fatty acid synthase (FAS) in mice fed a standard chow diet, vehicle treated mice fed a Paigen diet, and Compound A treated mice fed a Paigen diet.

FIG. 7 shows the liver expression level of small heterodimer partner (SHP), bile salt export pump (BSEP), and multiple drug resistance-2 (MDR2) in mice fed a standard chow diet, vehicle treated mice fed a Paigen diet, and Compound A treated mice fed a Paigen diet.

FIG. 8 shows images of gallbladders from vehicle treated or Compound A treated mice fed a Paigen diet.

FIG. 9 shows the serum level of serum aspartate aminotransferase (AST) activity in vehicle treated mice fed a standard chow diet, vehicle treated mice fed a methionine/choline deficient (MCD) diet, and Compound A treated mice fed a MCD diet.

FIG. 10 shows the serum level of murine keratinocyte-derived chemokine (mKC) in vehicle treated mice fed a standard chow diet, vehicle treated mice fed a MCD diet, and Compound A treated mice fed a MCD diet.

FIG. 11A shows the liver expression level of VCAM-1 and FIG. 11B shows the level of MCP-1 in vehicle treated mice fed a standard chow diet, vehicle treated mice fed a MCD diet, and Compound A treated mice fed a MCD diet.

FIG. 12A shows the liver expression level of tissue inhibitor of metalloproteinase-1 (TIMP-1) and FIG. 12B shows the level of matrix metalloproteinase-9 (MMP-9) and matrix metalloproteinase-14 (MMP-14) in vehicle treated mice fed a standard chow diet, vehicle treated mice fed a MCD diet, and Compound A treated mice fed a MCD diet.

FIG. 13 shows representative images of liver sections from mice fed a standard chow diet (control), vehicle treated mice fed a MCD diet, and Compound A treated mice fed a MCD diet. Sections were stained with Oil Red O.

FIG. 14 shows representative images of liver sections from mice fed a standard chow diet (control), vehicle treated mice fed a MCD diet, and Compound A treated mice fed a MCD diet. Sections were stained with H&E.

FIG. 15 shows representative images of liver sections from mice fed a standard chow diet (control), vehicle treated mice fed a MCD diet, and Compound A treated mice fed a MCD diet. Sections were stained with Trichrome.

FIG. 16 shows the liver expression level of CYP2E1 in vehicle or Compound A treated mice fed a standard chow diet and vehicle or Compound A treated mice fed a MCD diet.

FIG. 17A shows the liver expression level of FXR, FIG. 17B shows the liver expression level of SHP, and FIG. 17C shows the liver expression level of BSEP in vehicle treated mice fed a standard chow diet, and vehicle or Compound A treated mice fed a MCD diet.

FIG. 18 shows the serum level of ALT activity in wildtype (WT) and FXR deficient (FXRKO) mice fed a standard chow diet, vehicle treated WT and FXRKO mice fed a MCD diet, and Compound A treated WT and FXRKO mice fed a MCD diet.

FIG. 19 shows the gene expression level of VCAM-1 in the livers of WT and FXRKO mice fed a standard chow diet, vehicle treated WT and FXRKO mice fed a MCD diet, and Compound A treated WT and FXRKO mice fed a MCD diet.

FIG. 20A shows the gene expression level of tissue inhibitor of metalloproteinase-1 (TIMP-1), and FIG. 20B shows the expression level of collagen, type I, alpha 2 (Col1a2), in the livers of WT and FXRKO mice fed a standard chow diet, vehicle treated WT and FXRKO mice fed a MCD diet, and Compound A treated WT and FXRKO mice fed a MCD diet.

FIG. 21A shows the serum level of ALT activity and FIG. 21B shows the serum level of aspartate aminotransferase (AST) activity in mice fed a standard chow diet (WT/Chow), mice fed a MCD diet for 2 weeks (WT/MCD 2 w), 2-week vehicle treated mice fed a MCD diet for a total of 4 weeks (WT/MCD 4 w/V-2 w), 2-week Compound A treated mice fed a MCD diet for a total of 4 weeks (WT/MCD 4 w/Compound A-2 w), 4-week vehicle treated mice fed a MCD diet for a total of 6 weeks (WT/MCD 6 w/V-4 w), and 4-week Compound A treated mice fed a MCD diet for a total of 6 weeks (WT/MCD6 w/Compound A-4 w).

FIG. 22A shows the gene expression level of VCAM-1 and FIG. 22B shows the gene expression level of MCP-1 in the livers of mice fed a standard chow diet (WT/Chow), mice fed a MCD diet for 2 weeks (WT/MCD 2 w), 2-week vehicle treated mice fed a MCD diet for a total of 4 weeks (WT/MCD 4 w/V-2 w), 2-week Compound A treated mice fed a MCD diet for a total of 4 weeks (WT/MCD4 w/Compound A-2 w), 4-week vehicle treated mice fed a MCD diet for a total of 6 weeks (WT/MCD 6 w/V-4 w), and 4-week Compound A treated mice fed a MCD diet for a total of 6 weeks (WT/MCD6 w/Compound A-4 w).

FIG. 23A shows the gene expression level of Col1a2, FIG. 23B shows the gene expression level of MMP-2, and FIG. 23C shows the gene expression level of TIMP-1 in the livers of WT mice fed a standard chow diet (WT/Chow), mice fed a MCD diet for 2 weeks (WT/MCD 2 w), 2-week vehicle treated mice fed a MCD diet for a total of 4 weeks (WT/MCD 4 w/V-2 w), 2-week Compound A treated mice fed a MCD diet for a total of 4 weeks (WT/MCD4 w/Compound A-2 w), 4-week vehicle treated mice fed a MCD diet for a total of 6 weeks (WT/MCD 6 w/V-4 w), and 4-week Compound A treated mice fed a MCD diet for a total of 6 weeks (WT/MCD6 w/Compound A-4 w).

FIGS. 24A, 24B, and 24C show the levels of C-reactive protein (CRP) secretion (pg/ml) in Hep3B cells after stimulation with IL-6 (10 ng/ml), or IL-6 (50 ng/ml), or IL-6 (10 ng/ml & IL-1β 20 ng/ml), and treated with vehicle (DMSO) or Compound A (5 μM).

FIG. 25 shows the results of an experiment to determine the IC₅₀ of Compound A's inhibitory effect on CRP secretion in Hep3B cells treated with 10 ng/ml of IL-6.

FIGS. 26A and 26B show the CRP gene expression level in Hep3B cells stimulated with IL-6 (10 ng/ml or 50 ng/ml), and treated with vehicle (DMSO) or Compound A (1 μM).

FIG. 27 shows that FXR siRNA blocked Compound A's inhibitory effect on CRP secretion in Hep3B cells. The cells were transfected with FXR siRNA or control siRNA, stimulated with 50 ng/ml IL-6, and treated with control (DMSO) or Compound A (1 μM). CRP concentrations in the conditional media were measured by ELISA.

FIG. 28 shows the CRP and FXR relative gene expression levels in Hep3B cells. The Hep3B cells were transfected with FXR siRNA or control siRNA, stimulated with 50 ng/ml IL-6, and treated with control (DMSO) or Compound A (1 μM).

FIG. 29A shows the gene expression level of serum amyloid A-3 (SAA-3), FIG. 29B shows the serum amyloid P (SAP) level, and FIG. 29C shows the VCAM-1 level in the livers of WT and FXRKO mice, vehicle treated WT and FXRKO mice challenged with LPS, and Compound A treated WT and FXRKO mice challenged with LPS.

FIG. 30A shows the serum level of ALT activity and FIG. 30B shows serum level of AST activity in WT mice, vehicle treated WT mice challenged with CCl4, and Compound A treated WT mice challenged with CCl4. The data are presented as units per liter (U/L).

FIG. 31A shows the gene expression level of α smooth muscle actin (a-SMA) mRNA and FIG. 31B shows the gene expression level of transforming growth factor β1 (TGF-β1) mRNA in the livers of the WT mice, vehicle treated WT mice challenged with CCl4, and Compound A treated WT mice challenged with CCl4.

FIG. 32A shows the gene expression level of TIMP-1 and FIG. 32B shows the gene expression level of MMP-9 in the livers of the WT mice, vehicle treated WT mice challenged with CCl4, and Compound A treated WT mice challenged with CCl4.

As used in the present specification, the following words and phrases are generally intended to have the meanings as set forth below, except to the extent that the context in which they are used indicates otherwise.

As used herein “nonalcoholic fatty liver disease (NAFLD)” refers to a metabolic fatty liver disease occurring in the absence of alcohol abuse. In some embodiments, NAFLD is characterized by at least one of steatosis (simple fatty liver), nonalcoholic steatohepatitis (NASH), NAFLD induced hepatitis (inflammation), NAFLD induced fibrosis, and NAFLD induced cirrhosis. In some embodiments, NAFLD is characterized by features including neutral lipid deposition, intracellular lipid droplet formation, Kuppfer cell activation, inflammatory cell infiltration, inflammatory cholangitis, portal inflammation, fibrosis, oxidative stress, and acute phase response in the liver. In some embodiments, symptoms of NAFLD include fatigue, abdominal pain, lack of appetite, nausea, jaundice, intestinal bleeding, esophageal bleeding, and swelling in the extremities.

As used herein “cholesterol gallstone disease” refers to gallbladder disease with cholesterol precipitation in the bile. Cholesterol gallstone disease can be characterized by supersaturation of bile with cholesterol, precipitation of cholesterol crystals in the gallbladder, increased bile salt hydrophobicity, and gallbladder inflammation. Disrupted homeostasis of the bile components, cholesterol, bile salt, and phospholipids are thought to cause precipitation of cholesterol crystals. Normally, cholesterol is solubilized in mixed micelles together with bile salts and phospholipids. Under supersaturated cholesterol conditions, monohydrate crystals can enucleate from cholesterol-enriched vesicles, aggregate, fuse, and eventually precipitate into larger pathogenic crystals which can lead to gallstone formation. Gallstones can block the normal flow of bile in at least one duct system selected from the hepatic ducts, cystic ducts, and common bile ducts and the flow of digestive enzymes in the pancreatic duct. “Biliary symptoms,” as used herein, refer to symptoms caused by cholesterol gallstone disease. For example, these symptoms can include without limitation pain, nausea, vomiting, gastrointestinal symptoms (bloating, food intolerance, colic, gas, and indigestion), and gallbladder inflammation.

As used herein, “highly symptomatic” refers to having an incapacitative form of a disease. For example, a patient with cholesterol gallstone disease who is highly symptomatic can in some embodiments have one or more of large gallstones, numerous gallstones, and multiple symptoms of cholesterol gallstone disease.

In some embodiments, cholesterol gallstone disease is associated with NAFLD. Metabolic syndrome or Syndrome X, a collection of risk factors for cardiovascular disease, including for example and without limitation, obesity, altered lipid homeostasis, insulin resistance, and hyperglycemia, has been implicated in the development of both disorders, and the prevalence of cholesterol gallstone disease in NAFLD is higher than it is in the general population. In some embodiments, cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress, and acute phase response in the liver and an elevated level of at least one of vascular cell adhesion molecule-1 (VCAM-1), intercellular adhesion molecule-1 (ICAM-1), tumor necrosis factor α (TNFα), monocyte chemotactic protein-1 (MCP-1), keratinocyte-derived chemokine (KC), tissue inhibitor of metalloproteinase-1 (TIMP-1), collagen, type 1, alpha 2 (Col1a2), transforming growth factor β (TGF-β), α smooth muscle actin (a-SMA), at least one matrix metalloproteinase (MMP), at least one positive acute phase protein, alanine aminotransferase (ALT), aspartate aminotransferase (AST), Cytochrome P450 2E1 (CYP2E1), and cytokeratin 18 (CK-18). In some embodiments, the at least one MMP is selected from MMP-2, MMP-9, and MMP-14. In some embodiments, the at least one positive acute phase protein is selected from C-reactive protein (CRP), amyloid P component (SAP), and at least one serum amyloid A (SAA). In some embodiments, the at least one SAA is SAA-3.

As used herein, “Kuppfer cell activation” refers to the events that trigger Kuppfer cell activation and the resulting activities of Kuppfer cells, immune cells which reside in the liver. In some embodiments, endotoxin triggers Kuppfer cell activation. In some embodiments, the resulting activities of Kuppfer cells are cytokine and reactive oxygen species production and lead to inflammation and liver damage.

As used herein, reference to “inflammation” refers to basic reactions of the body to infection or irritation. In some embodiments, inflammation is manifested in hyperemia, edema, and infiltration of immune cells including, for example and without limitation, white blood cells, neutrophils, and macrophages. Inflammation in NAFLD can, for example, refer to hepatitis, inflammatory cell infiltration, inflammatory cholangitis, and portal inflammation in the liver. In some embodiments, inflammation results in an acute phase response. In cholesterol gallstone disease, inflammation can refer to gallbladder inflammation (cholecystitis) caused by gallstones.

As used herein, “treating” refers to any manner in which at least one symptom or feature of a disease or disorder is beneficially altered so as to delay the onset, retard the progression, or ameliorate the symptoms of the disease or disorder. In some embodiments, an existing disease is treated. In some embodiments, a patient who has not yet manifested a symptom or feature of a disease or disorder is treated. In some embodiments, a patient who has not yet manifested a symptom or feature of a disease or disorder, but who has manifested at least one risk factor for development of the disease or disorder is treated. In some embodiments the at least one risk factor is a the presence of a genotypic marker of predisposition to development of the disease or disorder. Treating nonalcoholic fatty liver disease with a FXR agonist, for example, can reduce at least one feature of the disease including neutral lipid deposition, intercellular lipid droplet formation, Kuppfer cell activation, inflammatory cell infiltration, inflammatory cholangitis, portal inflammation, fibrosis, oxidative stress, and acute phase response in the liver. In some embodiments, treating nonalcoholic fatty liver disease with a FXR agonist reduces the level of at least one of an inflammatory mediator involved in inflammation, for example but not limited to vascular cell adhesion molecule-1 (VCAM-1), intercellular adhesion molecule-1 (ICAM-1), tumor necrosis factor α (TNFα), monocyte chemotactic protein-1 (MCP-1), keratinocyte-derived chemokine (KC), and transforming growth factor β (TGF-β). In some embodiments, treating nonalcoholic fatty liver disease with a FXR agonist reduces the level of liver fibrosis markers, including, for example and without limitation, alanine aminotransferase (ALT), aspartate aminotransferase (AST), collagen, type 1, alpha 2 (Col1a2), alpha-smooth muscle actin (a-SMA), and TGF-β. In some embodiments, treating nonalcoholic fatty liver disease or fibrosis with a FXR agonist modulates the level of tissue inhibitor of metalloproteinase 1 (TIMP-1).

As used herein, “preventing” refers to administration of an agent to a patient so as to prevent the patient from developing a disease or disorder. In some embodiments prevention is measured over a finite period of time such as one month, three months, six months, one year, five years, ten years, or longer.

In some embodiments, the individual or relative levels of ALT and AST are measured to monitor liver damage or fibrosis. In some embodiments, the serum levels of ALT and AST are measured. In some embodiments, treating nonalcoholic fatty liver disease with a FXR agonist reduces the serum level of cytokeratin 18 (CK-18). CK-18 protein may be proteolytically cleaved by enzymes including caspases. In some embodiments, the level of CK-18 is assayed by measuring the level of intact CK-18 protein. In some embodiments, the level of CK-18 is assayed by measuring the level of one or more CK-18 proteolysis products.

In some embodiments, treating nonalcoholic fatty liver disease or fibrosis with a FXR agonist reduces the level of at least one MMP. In some embodiments, the at least one MMP is selected from MMP-2, MMP-9, and MMP-14.

In some embodiments, treating nonalcoholic fatty liver disease with a FXR agonist reduces the level of a positive acute phase protein, including, for example and without limitation, C-reactive protein (CRP), serum amyloid P component (SAP), and at least one serum amyloid A (SAA). In some embodiments, the at least one SAA is SAA-3.

In some embodiments, treating nonalcoholic fatty liver disease or oxidative stress with a FXR agonist reduces the level of CYP2E1, a reactive oxygen species generating microsomal enzyme. In some embodiments, treating nonalcoholic fatty liver disease elevates the level of at least one FXR target selected from fatty acid synthase (FAS), small heterodimer partner (SHP), bile salt export pump (BSEP), and multiple drug resistance-2 (MDR2).

Treating cholesterol gallstone disease with a FXR agonist can, for example, reduce a feature of the disease including at least one of gallstone incidence, biliary symptoms, and gallbladder inflammation. In some embodiments, treating cholesterol gallstone disease with a FXR agonist reduces at least one of gallstone incidence, gallstone dissolution time, bile cholesterol levels, and bile/salt phospholipid levels. In some embodiments, treating cholesterol gallstone disease with a FXR agonist reduces at least one symptom or feature of associated nonalcoholic fatty liver disease.

As used herein the phrase “therapeutically effective amount” refers to the amount sufficient to provide a therapeutic outcome regarding at least one symptom or feature of a disease or condition.

As used herein, the term “farnesoid X receptor (FXR)” refers to all mammalian forms of such receptor including, for example, alternative splice isoforms and naturally occurring isoforms (see, e.g. Huber et al, Gene (2002), Vol. 290, pp.: 35-43). Representative farnesoid X receptor species include, without limitation the rat (GenBank Accession No. NM_{—}021745), mouse (GenBank Accession No. NM_{—}009108), and human (GenBank Accession No. NM_{—}005123) forms of the receptor.

As used herein, “agent” refers to a substance including, but not limited to a chemical compound, such as a small molecule or a complex organic compound, a protein, such as an antibody or antibody fragment or a protein comprising an antibody fragment, or a genetic construct which acts at the DNA or mRNA level in an organism.

As used herein, reference to “modulate” refers to changing or altering an activity, function, or feature. The term “modulator” refers to an agent which modulates an activity, function, or feature. For example, an agent may modulate an activity by increasing or decreasing the activity compared to the effects on the activity in the absence of the agent. In some embodiments, a modulator is an agonist.

As used herein, the term “agonist” refers to an agent that triggers a response that is at least one response or partial response triggered by binding of an endogenous ligand of the receptor to the receptor. In some embodiments, the agonist acts directly or indirectly on a second agent that itself modulates the activity of the receptor. In some embodiments, the at least one response of the receptor is an activity of the receptor that can be measured with assays including but not limited to physiological, pharmacological, and biochemical assays. Exemplary assays include but are not limited to assays that measure the binding of an agent to the receptor, the binding of the receptor to a substrate such as but not limited to a nuclear receptor and a regulatory element of a target gene, the effect on gene expression assayed at the mRNA or resultant protein level, and the effect on an activity of proteins regulated either directly or indirectly by the receptor. For example, farnesoid X receptor activity can be measured by monitoring the level of at least one of VCAM-1, ICAM-1, TNFα, MCP-1, KC, ALT, AST, CK-18, TIMP-1, TGF-β, a-SMA, at least one MMP, at least one positive acute phase protein, CYP2E1, FAS, SHP, BSEP, and MDR2. In some embodiments, the at least one MMP is selected from MMP-2, MMP-9, and MMP-14. In some embodiments, the at least one positive acute phase protein is selected from CRP, SAP, and at least one SAA. In some embodiments, the at least one SAA is SAA-3.

As used herein, a reference to “level” of a factor refers to the expression of a polynucleotide or gene encoding the factor or to the activity of the protein corresponding to the factor. Expression of a polynucleotide or gene can refer to the production of a RNA transcript (mRNA) or the production of a protein, so the level of a factor can be measured by assaying the amounts of mRNA or protein produced. The level of a factor can also be measured by assaying the amount of activity of the protein produced. In some embodiments, the protein corresponding to the factor is a proteolytically cleaved protein. In some embodiments, the level of a factor is measured by assaying the amount of the proteolytically cleaved protein. For example, CK-18 may be proteolytically cleaved. Measuring the level of CK-18 may include measuring the amount of full-length CK-18 or the amount of at least one proteolytically cleaved form of CK-18.

As used herein, the term “inflammatory mediator” refers to a factor involved in inflammation. In some embodiments, an inflammatory mediator can promote proliferation, growth, survival, differentiation, or migration of cells involved in inflammation. Inflammatory mediators include for example and without limitation VCAM-1, ICAM-1, TNFα, MCP-1, TGF-β, and KC.

As used herein, “vascular cell adhesion molecule-1 (VCAM-1)” refers to all mammalian forms of the protein including, for example, alternative splice isoforms and naturally occurring isoforms. Representative VCAM-1 species include, without limitation the human variant 1 (GenBank Accession No. NM_{—}001078), human variant 2 (GenBank Accession No. NM_{—}080682), mouse (GenBank Accession No. NM_{—}011693) and rat (GenBank Accession No. NM_{—}012889) forms.

As used herein, “intercellular adhesion molecule-1 (ICAM-1)” refers to all mammalian forms of the protein including, for example, alternative splice isoforms and naturally occurring isoforms. Representative ICAM-1 species include, without limitation the human (GenBank Accession No. NM_{—}000201), mouse (GenBank Accession No. NM_{—}010493) and rat (GenBank Accession No. NM_{—}012967) forms.

As used herein, “tumor necrosis factor α (TNFα)” refers to all mammalian forms of the protein, including for example, alternative splice isoforms and naturally occurring isoforms. Representative TNFα species include, without limitation the human (GenBank Accession No. NM_{—}000594), mouse (GenBank Accession No. NM_{—}013693) and rat (GenBank Accession No. NM_{—}012675) forms.

As used herein, “monocyte chemotactic protein-1 (MCP-1),” also known as chemokine (C-C motif) ligand 2 (CCL2), refers to all mammalian forms of the protein, including for example, alternative splice isoforms and naturally occurring isoforms. Representative MCP-1 species include, without limitation, the human (GenBank Accession No. NM_{—}002982), mouse (GenBank Accession No. NM_{—}011333) and rat (GenBank Accession No. NM_{—}031530) forms.

As used herein, “keratinocyte-derived chemokine (KC),” also known as chemokine (C-X-C motif) ligand 1 refers to all mammalian forms of the protein, including for example, alternative splice isoforms and naturally occurring isoforms. As used herein, “murine keratinocyte-derived chemokine (mKC)” refers to all the murine forms of KC, including for example, alternative splice isoforms and naturally occurring isoforms. Representative KC species include, without limitation, the human (GenBank Accession No. NM_{—}001511), mouse (GenBank Accession No. NM_{—}008116) and rat (GenBank Accession No. NM_{—}030845) forms.

As used herein, “alanine aminotransferase (ALT)” refers to all mammalian forms of the protein, including for example, alternative splice isoforms and naturally occurring isoforms. Representative ALT species include, without limitation the human (GenBank Accession No. NM_{—}005309), mouse (GenBank Accession No. NM_{—}182805) and rat (GenBank Accession No. NM_{—}031039) forms.

As used herein, “aspartate aminotransferase (AST),” also known as glutamic-oxaloacetic transaminase (GOT), refers to all mammalian forms of the protein, including for example, alternative splice isoforms and naturally occurring isoforms. AST can refer to at least one of AST1, also known as GOT1 and AST2, also known as GOT2. Representative AST1 species include, without limitation, the human (GenBank Accession No. NM_{—}002079), mouse (GenBank Accession No. NM_{—}010324) and rat (GenBank Accession No. NM_{—}012571) forms. Representative AST2 species include, without limitation, the human (GenBank Accession No. NM_{—}002080), mouse (GenBank Accession No. NM_{—}0103225) and rat (GenBank Accession No. NM_{—}013177) forms.

As used herein, “tissue inhibitor of metalloproteinase-1 (TIMP-1)” refers to all mammalian forms of the protein, including for example, alternative splice isoforms and naturally occurring isoforms. Representative TIMP-1 species include, without limitation, the human (GenBank Accession No. NM_{—}003254), mouse (GenBank Accession No. NM_{—}011593) and rat (GenBank Accession No. NM_{—}053819) forms.

As used herein, “MMP” refers to a member of the matrix metalloproteinase family. There are at least twenty-five known members of the MMP family. In certain embodiments, a MMP is at least one of MMP-2, MMP-9, and MMP-14.

As used herein, “matrix metalloproteinase-2 (MMP-2)” refers to all mammalian forms of the protein, including, for example, alternative splice isoforms and naturally occurring isoforms. Representative MMP-2 species include, without limitation the human variant 1 (GenBank Accession No. NM_{—}004530), the human variant 2 (GenBank Accession No. NM_{—}001127891), mouse (GenBank Accession No. NM_{—}008610), and rat (GenBank Accession No. NM_{—}031054) forms.

As used herein, “matrix metalloproteinase-9 (MMP-9)” refers to all mammalian forms of the protein, including for example, alternative splice isoforms and naturally occurring isoforms. Representative MMP-9 species include, without limitation the human (GenBank Accession No. NM_{—}004994), mouse (GenBank Accession No. NM_{—}013599), and rat (GenBank Accession No. NM_{—}031055) forms.

As used herein, “matrix metalloproteinase-14 (MMP-14)” refers to all mammalian forms of the protein, including for example, alternative splice isoforms and naturally occurring isoforms. Representative MMP-14 species include, without limitation, the human (GenBank Accession No. NM_{—}004995), mouse (GenBank Accession No. NM_{—}008608) and rat (GenBank Accession No. NM_{—}031056) forms.

As used herein, “collagen, type 1, alpha 2 (Col1a2)” refers to all mammalian forms of the protein, including for example, alternative splice isoforms and naturally occurring isoforms. Representative Col1a2 species include, without limitation the human (GenBank Accession No. NM_{—}000089), mouse (GenBank Accession No. NM_{—}007743), and rat (GenBank Accession No. NM_{—}053356) forms.

As used herein, “α smooth muscle actin (a-SMA)” refers to all mammalian forms of the protein, including for example, alternative splice isoforms and naturally occurring isoforms. Representative a-SMA species include, without limitation the human (GenBank Accession No. NM_{—}001613), mouse (GenBank Accession No. NM_{—}007392), and rat (GenBank Accession No. NM_{—}031004) forms.

As used herein, “transforming growth factor β (TGF-β)” refers to all mammalian forms of the protein, including for example, alternative splice isoforms and naturally occurring isoforms. Representative TGF-β species include the isoforms TGF-β1, TGF-β2, and TGF-β3.

As used herein, “TGF-β1” refers to all mammalian forms of the protein, including for example, alternative splice isoforms and naturally occurring isoforms. Representative TGF-β1 species include, without limitation the human (GenBank Accession No. NM_{—}000660), mouse (GenBank Accession No. NM_{—}011577), and rat (GenBank Accession No. NM_{—}021578) forms.

As used herein, “TGF-β2” refers to all mammalian forms of the protein, including for example, alternative splice isoforms and naturally occurring isoforms. Representative TGF-β2 species include, without limitation the human (GenBank Accession No. NM_{—}003238), mouse (GenBank Accession No. NM_{—}009367), and rat (GenBank Accession No. NM_{—}031131) forms.

As used herein, “TGF-β3” refers to all mammalian forms of the protein, including for example, alternative splice isoforms and naturally occurring isoforms. Representative TGF-β3 species include, without limitation the human (GenBank Accession No. NM_{—}003239), mouse (GenBank Accession No. NM_{—}009368), and rat (GenBank Accession No. NM_{—}013174) forms.

As used herein, the “acute phase response” refers to a systemic reaction in organisms to local or systemic disturbances in its homeostasis caused by infection, tissue injury, trauma, cancer, or disorders, such as and not limited to NAFLD or liver fibrosis or liver inflammation. In some embodiments, an acute phase response is induced by lipopolysaccharide or by a chemical such as and not limited to carbon tetrachloride (CCl4). In the acute phase response, pro-inflammatory cytokines are released, activating the vascular system and inflammatory cells. The pattern of liver gene and protein expression can then change. In some embodiments, expression of acute phase proteins are modulated in an acute phase response. In some embodiments, the level of an acute phase protein is measured to determine the level of an acute phase response. In some embodiments, an increase in an acute phase response causes at least one of an increase in the level of at least one a positive acute phase protein or a decrease in the level of at least one negative acute phase protein.

As used herein, an “acute phase protein” refers to at least one protein whose expression can change during the acute phase response. Acute phase proteins whose level can increase during the response are referred to as positive acute phase proteins. Positive acute phase proteins include, for example and without limitation, C-reactive protein (CRP), at least one serum amyloid A (SAA), and serum amyloid P component (SAP). In some embodiments, the at least one SAA is SAA-3. Acute phase proteins whose level can decrease during the response are referred to as negative acute phase proteins. A negative acute phase protein includes, for example and without limitation, transferrin, albumin, and retinol binding protein.

As used herein, “CRP” refers to refers to the mammalian forms of the protein, including for example, alternative splice isoforms and naturally occurring isoforms, which are acute phase proteins. In some embodiments, CRP is a positive acute phase protein in human, but the murine forms of CRP protein are not acute phase proteins. Representative CRP species include, without limitation the human (GenBank Accession No. NM_{—}000567).

As used herein, “SAP” refers to all mammalian forms of the serum amyloid P component protein, including for example, alternative splice isoforms and naturally occurring isoforms. Representative SAP species include, without limitation the human (GenBank Accession No. NM_{—}001639), mouse (GenBank Accession No. NM_{—}011318), and rat (GenBank Accession No. NM_{—}017170) forms.

As used herein, “SAA” refers to a member of the serum amyloid A family. There are at least four members of the SAA family. In some embodiments, a SAA is SAA-3. As used herein, “SAA-3” refers to all mammalian forms of the protein, including for example, alternative splice isoforms and naturally occurring isoforms. Representative SAA-3 species include, without limitation the mouse (GenBank Accession No. NM_{—}011315) form.

As used herein, “Cytochrome P450 2E1 (CYP2E1)” refers to all mammalian forms of the protein, including for example, alternative splice isoforms and naturally occurring isoforms. Representative CYP2E1 species include, without limitation, the human (GenBank Accession No. NM_{—}000773), mouse (GenBank Accession No. NM_{—}021282) and rat (GenBank Accession No. NM_{—}031543) forms.

As used herein, “cytokeratin 18 (CK-18),” encoded by the keratin 18 (KRT18) gene, refers to all mammalian forms of the protein, including for example, alternative splice isoforms, naturally occurring isoforms, and proteolytically cleaved forms. Representative CK-18 species include, without limitation, the human variant 1 (GenBank Accession No. NM_{—}000224), human variant 2 (GenBank Accession No. NM_{—}199187), and mouse (GenBank Accession No. NM_{—}010664) forms.

As used herein, “FXR target” refers to a factor that is regulated by FXR activity. In some embodiments, FXR activity regulates transcription of a gene encoding a FXR target. For example and without limitation, FXR target genes include the genes encoding fatty acid synthase (FAS), small heterodimer partner (SHP), bile salt export pump (BSEP), and multiple drug resistance-2 (MDR2).

As used herein, “fatty acid synthase (FAS)” refers to all mammalian forms of the protein, including for example, alternative splice isoforms and naturally occurring isoforms. Representative FAS species include, without limitation the human (GenBank Accession No. NM_{—}004104), mouse (GenBank Accession No. NM_{—}007988) and rat (GenBank Accession No. NM_{—}017332) forms.

As used herein, the term “small heterodimer partner (SHP)” refers to all mammalian forms of the protein, including for example, alternative splice isoforms and naturally occurring isoforms. Representative SHP species include, without limitation the human (GenBank Accession No. NM_{—}021969), mouse (GenBank Accession No. NM_{—}011850), and rat (GenBank Accession No. NM_{—}057133) forms.

As used herein, “bile salt export pump (BSEP)”, also known as ATP-binding cassette, sub-family B (MDR/TAP), member 11 (ABCB1), refers to all mammalian forms of the protein, including for example, alternative splice isoforms and naturally occurring isoforms. Representative BSEP species include, without limitation the human (GenBank Accession No. NM_{—}003742), mouse (GenBank Accession No. NM_{—}021022) and rat (GenBank Accession No. NM_{—}031760) forms.

As used herein, “multiple drug resistance-2 (MDR-2)”, also known as ATP-binding cassette, sub-family B (MDR/TAP), member 4 (ABCB4), refers to all mammalian forms of the protein, including for example, alternative splice isoforms and naturally occurring isoforms. Representative MDR-2 species include, without limitation the human (GenBank Accession No. NM_{—}000443), mouse (GenBank Accession No. NM_{—}008830) and rat (GenBank Accession No. NM_{—}012690) forms.

As used herein, the term “coadministering” refers to a dosage regimen for a first agent that overlaps with the dosage regimen of a second agent, or to simultaneous administration of the first agent and the second agent. A dosage regimen is characterized by dosage amount, frequency, and duration. Two dosage regimens overlap if between a first and a second administration of a first agent the second agent is administered.

As used herein, the phrase “effective amount” refers to the amount sufficient to increase or reduce a specified activity, function, or feature.

Provided are methods of treating nonalcoholic fatty liver disease (NAFLD) in a patient. The methods include administering to the patient a therapeutically effective amount of at least one farnesoid X receptor (FXR) agonist. In some embodiments, the nonalcoholic fatty liver disease is characterized by at least one of steatosis, nonalcoholic steatohepatitis (NASH), NAFLD induced hepatitis, NAFLD induced fibrosis, and NAFLD induced cirrhosis. In some embodiments, the at least one FXR agonist reduces at least one feature of nonalcoholic fatty liver disease selected from neutral lipid deposition, intracellular lipid droplet formation, Kupffer cell activation, inflammatory cell infiltration, inflammatory cholangitis, portal inflammation, fibrosis, oxidative stress, and acute phase response in the liver. In some embodiments, administration of the at least one FXR agonist to the patient causes at least one of a reduction in the level of at least one of vascular cell adhesion molecule-1 (VCAM-1), intercellular adhesion molecule-1 (ICAM-1), tumor necrosis factor α (TNFα), monocyte chemotactic protein-1 (MCP-1), keratinocyte-derived chemokine (KC), collagen, type 1, alpha 2 (Col1a2), transforming growth factor β (TGF-β), a smooth muscle actin (a-SMA), at least one matrix metalloproteinase (MMP), at least one positive acute phase protein, and Cytochrome P450 2E1 (CYP2E1) and a modulation in the level of tissue inhibitor of metalloproteinase 1 (TIMP-1) in the patient. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments the at least one SAA is SAA-3. In some embodiments, the at least one FXR agonist reduces the serum level of at least one of alanine aminotransferase (ALT), aspartate aminotransferase (AST), cytokeratin 18 (CK-18) in the patient. In some embodiments, the at least one FXR agonist elevates the level of at least one FXR target in the patient selected from fatty acid synthase (FAS), small heterodimer partner (SHP), bile salt export pump (BSEP), and multiple drug resistance-2 (MDR2).

Also provided are methods of modulating the level of at least one of KC, ALT, AST, CK-18, Col1a2, TGF-β, a-SMA, at least one MMP, at least one positive acute phase protein, TIMP-1, and CYP2E1 in a patient. The methods include providing to the patient an effective amount of at least one FXR modulator, to thereby modulate the level of at least one of KC, ALT, AST, CK-18, Col1a2, TGF-β, a-SMA, at least one MMP, at least one positive acute phase protein, TIMP-1, and CYP2E1 in the patient. In some embodiments, the level of at least one of KC, ALT, AST, CK-18 Col1a2, TGF-β, a-SMA, at least one MMP, at least one positive acute phase protein, TIMP-1, and CYP2E1 is reduced in the patient and the at least one FXR modulator is a FXR agonist. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3.

Also provided are methods of identifying a FXR modulator. The methods include administering a test agent to a mammal; determining at least one of the following features of the mammal, in the presence or absence of the test agent: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver; and selecting a FXR modulator which modulates at least one of the following features of the mammal: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver. In some embodiments, the FXR modulator is a FXR agonist and the FXR agonist reduces at least one of the following features of the mammal: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver in the mammal.

Also provided are further methods of identifying a FXR modulator. The methods include administering a test agent to a mammal; determining the level of at least one of the following factors in the mammal, in the presence or absence of the test agent: TNFα, MCP-1, KC, ALT, AST, CK-18, Col1a2, TGF-β, a-SMA, at least one MMP, at least one positive acute phase protein, TIMP-1, and CYP2E1; and selecting a FXR modulator which modulates the level of at least one of the following factors in the mammal: TNFα, MCP-1, KC, ALT, AST, CK-18, Col1a2, TGF-β, a-SMA, at least one MMP, at least one positive acute phase protein, TIMP-1, and CYP2E1 in the mammal. In some embodiments, the FXR modulator is a FXR agonist and the FXR agonist reduces the level of at least one of the following factors in the mammal: TNFα, MCP-1, KC, ALT, AST, CK-18, Col1a2, TGF-β, a-SMA, at least one MMP, at least one positive acute phase protein, TIMP-1, and CYP2E1. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3.

Also provided are further methods of treating nonalcoholic fatty liver disease in a patient. The methods include administering to the patient a therapeutically effective amount of at least one FXR agonist. In some embodiments, the at least one FXR agonist is identified by administering a test agent to a mammal; determining at least one of the following features of the mammal in the presence or absence of the test agent: (a) neutral lipid deposition, (b) intracellular lipid droplet formation, (c) Kupffer cell activation, (d) inflammatory cell infiltration, (e) inflammatory cholangitis, portal inflammation, (g) fibrosis, (h) oxidative stress, and (i) acute phase response in the liver; and selecting a FXR agonist which reduces at least one of the following features of the mammal: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver.

Also provided are further methods of treating nonalcoholic fatty liver disease in a patient. The methods include administering to the patient a therapeutically effective amount of at least one FXR agonist. In some embodiments, the at least one FXR agonist is identified by incubating a test agent with a cell; determining the level of at least one of the following factors in the mammal in the presence or absence of the test agent: VCAM-1, ICAM-1, TNFα, MCP-1, KC, ALT, AST, CK-18, TIMP-1, Col1a2, TGF-β, a-SMA, at least one MMP, at least one positive acute phase protein, CYP2E1, FAS, SHP, BSEP, and MDR2; and selecting a FXR agonist which has at least one property selected from reducing the level of at least one of VCAM-1, ICAM-1, TNFα, MCP-1, KC, ALT, AST, CK-18, Col1a2, TGF-β, a-SMA, at least one MMP, at least one positive acute phase protein, and CYP2E1, modulating the level of TIMP-1, and elevating the level of at least one of FAS, SHP, BSEP, and MDR2 in the mammal. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3.

Also provided are methods of treating a patient with existing cholesterol gallstone disease. The methods include administering to the patient a therapeutically effective amount of at least one FXR agonist. In some embodiments, the patient is characterized by at least one feature selected from is highly symptomatic, is awaiting a cholecystectomy, and is not a suitable candidate for surgical intervention. In some embodiments, the at least one FXR agonist reduces at least one feature of cholesterol gallstone disease selected from gallstone incidence, gallstone dissolution time, bile cholesterol levels, bile salt/phospholipid ratios, biliary symptoms, and gallbladder inflammation in the patient. In some embodiments, the at least one FXR agonist reduces at least one feature selected from neutral lipid deposition, intracellular lipid droplet formation, Kupffer cell activation, inflammatory cell infiltration, inflammatory cholangitis, portal inflammation, fibrosis, oxidative stress, and acute phase response in the liver of the patient. In some embodiments, the administration of the at least one FXR agonist to the patient causes at least one of a reduction in the level of at least one of VCAM-1, ICAM-1, TNFα, MCP-1, KC, Col1a2, TGF-β, a-SMA, at least one MMP, at least one positive acute phase protein, and CYP2E1 and a modulation in the level of TIMP-1 in the patient. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3. In some embodiments, the at least one FXR agonist reduces the serum level of at least one of ALT, AST, and CK-18 in the patient. In some embodiments, the at least one FXR agonist elevates the level of at least one FXR target in the patient selected from FAS, SHP, BSEP, and MDR2. In some embodiments, the methods further include coadministering to the patient a therapeutically effective amount of ursodeoxycholic acid.

Also provided are methods of treating a patient with existing cholesterol gallstone disease, in which the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress, and acute phase response in the liver, and an elevated level of at least one of VCAM-1, ICAM-1, TNFα, MCP-1, KC, TIMP-1, Col1a2, TGF-β, a-SMA, at least one MMP, at least one positive acute phase protein, CYP2E1, ALT, AST, and CK-18. The methods include administering to the patient a therapeutically effective amount of at least one FXR agonist. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3. In some embodiments, the patient is characterized by at least one feature selected from is highly symptomatic, is awaiting a cholecystectomy, and is not a suitable candidate for surgical intervention. In some embodiments, the at least one FXR agonist reduces at least one feature of cholesterol gallstone disease selected from gallstone incidence, gallstone dissolution time, bile cholesterol levels, bile salt/phospholipid ratios, biliary symptoms, and gallbladder inflammation. In some embodiments, the at least one FXR agonist reduces at least one feature of cholesterol gallstone disease selected from neutral lipid deposition, intracellular lipid droplet formation, inflammatory cell infiltration, inflammatory cholangitis, portal inflammation, fibrosis, oxidative stress, and acute phase response in the liver of the patient. In some embodiments, the administration of the at least one FXR agonist to the patient causes at least one of a reduction in the level of at least one of VCAM-1, ICAM-1, TNFα, MCP-1, KC, Col1a2, TGF-β, a-SMA, at least one MMP, at least one positive acute phase protein, and CYP2E1 and a modulation in the level of TIMP-1 in the patient. In some embodiments, the at least one FXR agonist reduces the serum level of at least one of ALT, AST, and CK-18 in the patient. In some embodiments, the at least one FXR agonist elevates the level of at least one FXR target in the patient selected from FAS, SHP, BSEP, and MDR2. In some embodiments, the methods further include coadministering to the patient a therapeutically effective amount of ursodeoxycholic acid.

Also provided are further methods of identifying a FXR modulator. The methods include administering a test agent to a mammal; determining at least one of the following features in the mammal in the presence or absence of the test agent: (a) gallstone incidence, (b) gallstone dissolution time, (c) bile cholesterol lipids, (d) bile salt/phospholipid ratios, (e) biliary symptoms, and (f) gallbladder inflammation; and selecting a FXR modulator which modulates at least one of (a) gallstone incidence, (b) gallstone dissolution time, (c) bile cholesterol lipids, (d) bile salt/phospholipid ratios, (e) biliary symptoms, and (f) gallbladder inflammation in the mammal. In some embodiments, the FXR modulator is a FXR agonist and the FXR agonist reduces at least one of (a) gallstone incidence, (b) gallstone dissolution time, (c) bile cholesterol lipids, (d) bile salt/phospholipid ratios, (e) biliary symptoms, and (f) gallbladder inflammation in the mammal.

Also provided are further methods of treating a patient with existing cholesterol gallstone disease. The methods include administering to the patient a therapeutically effective amount of at least one FXR agonist. In some embodiments, the at least one FXR agonist is identified by administering a test agent to a mammal; determining at least one the following features in the mammal in the presence or absence of the test agent: (a) gallstone incidence, (b) gallstone dissolution time, (c) bile cholesterol lipids, (d) bile salt/phospholipid ratios, (e) biliary symptoms, and (f) gallbladder inflammation; and selecting a FXR agonist which reduces at least one of (a) gallstone incidence, (b) gallstone dissolution time, (c) bile cholesterol lipids, (d) bile salt/phospholipid ratios, (e) biliary symptoms, and (f) gallbladder inflammation in the mammal. In some embodiments, the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress in the liver, and an elevated level of at least one of VCAM-1, ICAM-1, TNFα, MCP-1, KC, TIMP-1, Col1a2, TGF-β, a-SMA, at least one MMP, at least one positive acute phase protein, CYP2E1, ALT, AST, and CK-18 in the mammal. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3.

Also provided are further methods of treating a patient with existing cholesterol gallstone disease. The methods include administering to the patient a therapeutically effective amount of at least one FXR agonist. In some embodiments, the at least one FXR agonist is identified by incubating a test agent with a cell; determining the level of at least one of the following factors in the presence or absence of the test agent: VCAM-1, ICAM-1, TNFα, MCP-1, KC, ALT, AST, CK-18, CYP2E1, TIMP-1, Col1a2, TGF-β, a-SMA, at least one MMP, at least one positive acute phase protein, FAS, SHP, BSEP, and MDR2; and selecting a FXR agonist with at least one property selected from reducing the level of at least one of VCAM-1, ICAM-1, TNFα, MCP-1, KC, ALT, AST, CK-18, CYP2E1, Col1a2, TGF-β, a-SMA, at least one MMP, at least one positive acute phase protein, modulating the level of TIMP-1, and elevating the level of at least one of FAS, SHP, BSEP, and MDR2. In some embodiments, the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress, and acute phase response in the liver, and an elevated level of at least one of VCAM-1, ICAM-1, TNFα, MCP-1, KC, TIMP-1, Col1a2, TGF-β, a-SMA, at least one MMP, at least one positive acute phase protein, CYP2E1, ALT, AST, and CK-18 in the mammal. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3.

Also provided are further methods of treating a patient with existing cholesterol gallstone disease. The methods include administering to the patient a therapeutically effective amount of at least one FXR agonist. In some embodiments, the at least one FXR agonist is identified by administering a test agent to a mammal; determining at least one of the following features of the mammal in the presence or absence of the test agent: (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver; and selecting a FXR agonist which reduces at least one of (a) neutral lipid deposition, (b) intracellular lipid droplet formation, (c) Kupffer cell activation, (d) inflammatory cell infiltration, (e) inflammatory cholangitis, (f) portal inflammation, (g) fibrosis, (h) oxidative stress, and (i) acute phase response in the liver of the mammal. In some embodiments, the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress, and acute phase response in the liver, and an elevated level of at least one of VCAM-1, ICAM-1, TNFα, MCP-1, KC, TIMP-1, Col1a2, TGF-β, a-SMA, at least one MMP, at least one positive acute phase protein, CYP2E1, ALT, AST, and CK-18 in the mammal. In some embodiments, the at least one MMP is selected from MMP-2, MMP-9, and MMP-14. In some embodiments, the at least one positive acute phase protein is selected from C-reactive protein (CRP), serum amyloid P component (SAP), and at least one serum amyloid A (SAA). In some embodiments, the at least one SAA is SAA-3.

Also provided are further methods of identifying a FXR modulator. The methods comprise incubating a test agent with a cell; determining the level of CRP in the presence or absence of the test agent; and selecting a FXR modulator which modulates the level of CRP. In some embodiments, the FXR modulator is a FXR agonist and the FXR agonist reduces the level of CRP.

In some embodiments provided herein the FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro4H-thieno[2,3-D]azepine-8-carboxylic acid ethyl ester;
6-(4-fluoro-benzoyl)-3,6,7,8-tetrahydro-imidazo[4,5-D]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3(4-fluorobenzoyl)-2-methyl-1,2,3,6-tetrahydroazepino[4,5-b]indole-5-carboxylate; and
n-propyl 3(4-fluorobenzoyl)-2-methyl-8-fluoro-1,2,3,6-tetrahydroazepino[4,5-b]indole-5-carboxylate.

In some embodiments of the methods the FXR agonist or modulator is selected from a compound disclosed in at least one of U.S. Patent Application Publication No. 2004/0023947A1, published Feb. 5, 2004, U.S. Patent Application Publication No. 2005/0054634A1, published Mar. 10, 2005, U.S. Patent Application Publication No. 2007/0015746A1, published Jan. 18, 2007, and International Patent Application Publication No. 2007/070796, published Jun. 21, 2007, each of which are hereby incorporated herein by reference in their entirety.

Pharmaceutical compositions for use in the methods herein are formulated to contain therapeutically effective amounts of at least one FXR modulator or pharmaceutically acceptable derivative. The pharmaceutical compositions are useful, for example, in the treatment of at least one of nonalcoholic fatty liver disease and cholesterol gallstone disease. Pharmaceutically acceptable derivatives include acids, bases, enol ethers and esters, salts, esters, hydrates, solvates and prodrug forms. The derivative is selected such that its pharmacokinetic properties are superior with respect to at least one characteristic to the corresponding neutral agent. The FXR modulator may be derivatized prior to formulation.

In some embodiments, the at least one FXR modulator or pharmaceutically acceptable derivative is formulated into a suitable pharmaceutical preparation such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the FXR modulator or pharmaceutically acceptable derivative is formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more FXR modulators or pharmaceutically acceptable derivatives are mixed with a suitable pharmaceutical carrier or vehicle.

The concentrations of the FXR modulator or pharmaceutically acceptable derivative in the compositions are effective for delivery of an amount, upon administration, that treats one or more of the symptoms of at least one of nonalcoholic fatty liver disease and cholesterol gallstone disease.

Typically, by way of example and without limitation, the compositions are formulated for single dosage administration. To formulate a composition, the weight fraction of the FXR modulator or pharmaceutically acceptable derivative is dissolved, suspended, dispersed or otherwise mixed in a selected vehicle at an effective concentration such that the treated condition, nonalcoholic fatty liver disease or cholesterol gallstone disease, is relieved or ameliorated. Pharmaceutical carriers or vehicles suitable for administration of the FXR modulator or pharmaceutically acceptable derivative include any such carriers known to those skilled in the art to be suitable for the particular mode of administration.

In addition, the FXR modulator or pharmaceutically acceptable derivative can be formulated as the sole modulator in the composition or can be combined with other modulators. Liposomal suspensions, including tissue-targeted liposomes, can also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) can be formed by drying down egg phosphatidyl choline and brain phosphatidyl serine (7:3 molar ratio) on the inside of a flask. A solution of a FXR modulator or pharmaceutically acceptable derivative provided herein in phosphate buffered saline lacking divalent cations (PBS) is added and the flask shaken until the lipid film is dispersed. The resulting vesicles are washed to remove unencapsulated FXR modulator or pharmaceutically acceptable derivative, pelleted by centrifugation, and then resuspended in PBS.

The FXR modulator or pharmaceutically acceptable derivative is included in the pharmaceutically acceptable carrier in an amount sufficient to exert a therapeutically useful effect in the absence of undesirable side effects on the patient treated. The therapeutically effective concentration may be determined empirically by testing the compositions in in vitro and in vivo systems described herein and in International Patent Application Publication Nos. 99/27365 and 00/25134 and then extrapolated there from for dosages for humans.

The concentration of the FXR modulator or pharmaceutically acceptable derivative in the pharmaceutical composition will depend on absorption, inactivation and excretion rates of the modulator, the physicochemical characteristics of the modulator, the dosage schedule, and amount administered as well as other factors known to those of skill in the art. For example, the amount that is delivered is sufficient to treat at least one of nonalcoholic fatty liver disease and cholesterol gallstone disease.

Typically a therapeutically effective dosage should produce a serum concentration of FXR modulator or pharmaceutically acceptable derivative of from about 0.1 ng/ml to about 50-100 μg/ml. The pharmaceutical compositions typically should provide a dosage of from about 0.001 mg to about 2000 mg of FXR modulator or pharmaceutically acceptable derivative per kilogram of body weight per day. Pharmaceutical dosage unit forms are prepared to provide from about 1 mg to about 1000 mg, such as from about 10 to about 500 mg of the FXR modulator or pharmaceutically acceptable derivative or a combination of modulators per dosage unit form.

The FXR modulator or pharmaceutically acceptable derivative may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed methods.

Thus, effective concentrations or amounts of one or more FXR modulators or pharmaceutically acceptable derivatives thereof are mixed with a suitable pharmaceutical carrier or vehicle for systemic, topical or local administration to form pharmaceutical compositions. FXR modulators or pharmaceutically acceptable derivatives are included in an amount effective for treating at least one of nonalcoholic fatty liver disease and cholesterol gallstone disease. The concentration of FXR modulator or pharmaceutically acceptable derivative in the composition will depend on absorption, inactivation, excretion rates of the FXR modulator or pharmaceutically acceptable derivative, the dosage schedule, amount administered, particular formulation as well as other factors known to those of skill in the art.

The compositions are intended to be administered by a suitable route, including by way of example and without limitation orally, parenterally, rectally, topically and locally. For oral administration, capsules and tablets can be used. The compositions are in liquid, semi-liquid or solid form and are formulated in a manner suitable for each route of administration.

Solutions or suspensions used for parenteral, intradermal, subcutaneous, or topical application can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampoules, disposable syringes or single or multiple dose vials made of glass, plastic or other suitable material.

In instances in which the FXR modulators or pharmaceutically acceptable derivatives exhibit insufficient solubility, methods for solubilizing the FXR modulators or pharmaceutically acceptable derivatives may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Pharmaceutically acceptable derivatives of the FXR modulators may be used in formulating effective pharmaceutical compositions.

Upon mixing or addition of the FXR modulator or pharmaceutically acceptable derivative(s), the resulting mixture may be a solution, suspension, emulsion or the like. The form of the resulting mixture depends upon a number of factors, including the intended mode of administration and the solubility of the FXR modulator or pharmaceutically acceptable derivative in the selected carrier or vehicle. The effective concentration is sufficient for treating one or more symptoms of at least one of nonalcoholic fatty liver disease and cholesterol gallstone disease and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the agents or pharmaceutically acceptable derivatives thereof. The FXR modulator or pharmaceutically acceptable derivative thereof is typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the FXR modulator or pharmaceutically acceptable derivative sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the FXR modulator or pharmaceutically acceptable derivative, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an modulator as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art; for example, see Remington: The Science and Practice of Pharmacy. 21^{st} Edition. Philadelphia, Pa. Lippincott Williams & Wilkins. 2005. The composition or formulation to be administered will, in any event, contain a quantity of the FXR modulator or pharmaceutically acceptable derivative in an amount sufficient to alleviate the symptoms of the treated subject.

Dosage forms or compositions containing FXR modulator or pharmaceutically acceptable derivative in the range of 0.005% to 100% with the balance made up from non-toxic carrier may be prepared. For oral administration, a pharmaceutically acceptable non-toxic composition is formed by the incorporation of any of the normally employed excipients, such as, for example and without limitation, pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% FXR modulator or pharmaceutically acceptable derivative, such as 0.1-85%, or such as 75-95%.

The FXR modulator or pharmaceutically acceptable derivative may be prepared with carriers that protect the modulator or pharmaceutically acceptable derivative against rapid elimination from the body, such as time release formulations or coatings. The compositions may include other modulators to obtain desired combinations of properties. FXR modulators or pharmaceutically acceptable derivatives thereof, may also be advantageously administered for therapeutic or prophylactic purposes together with another pharmacological agent or modulator known in the general art to be of value in treating at least one of nonalcoholic fatty liver disease and cholesterol gallstone disease.

Oral pharmaceutical dosage forms include, by way of example and without limitation, solid, gel and liquid. Solid dosage forms include tablets, capsules, granules, and bulk powders. Oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In some embodiments, the formulations are solid dosage forms, such as capsules or tablets. The tablets, pills, capsules, troches and the like can contain any of the following ingredients, or agents of a similar nature: a binder; a diluent; a disintegrating agent; a lubricant; a glidant; a sweetening agent; and a flavoring agent.

Examples of binders include, by way of example and without limitation, microcrystalline cellulose, gum tragacanth, glucose solution, acacia mucilage, gelatin solution, sucrose, and starch paste. Lubricants include, by way of example and without limitation, talc, starch, magnesium or calcium stearate, lycopodium and stearic acid. Diluents include, by way of example and without limitation, lactose, sucrose, starch, kaolin, salt, mannitol, and dicalcium phosphate. Glidants include, by way of example and without limitation, colloidal silicon dioxide. Disintegrating agents include, by way of example and without limitation, crosscarmellose sodium, sodium starch glycolate, alginic acid, corn starch, potato starch, bentonite, methylcellulose, agar and carboxymethylcellulose. Coloring agents include, by way of example and without limitation, any of the approved certified water soluble FD and C dyes, mixtures thereof; and water insoluble FD and C dyes suspended on alumina hydrate. Sweetening agents include, by way of example and without limitation, sucrose, lactose, mannitol and artificial sweetening agents such as saccharin, and any number of spray dried flavors. Flavoring agents include, by way of example and without limitation, natural flavors extracted from plants such as fruits and synthetic blends of agents which produce a pleasant sensation, such as, but not limited to peppermint and methyl salicylate. Wetting agents include, by way of example and without limitation, propylene glycol monostearate, sorbitan monooleate, diethylene glycol monolaurate, and polyoxyethylene laural ether. Emetic-coatings include, by way of example and without limitation, fatty acids, fats, waxes, shellac, ammoniated shellac and cellulose acetate phthalates. Film coatings include, by way of example and without limitation, hydroxyethylcellulose, sodium carboxymethylcellulose, polyethylene glycol 4000 and cellulose acetate phthalate.

If oral administration is desired, the FXR modulator or pharmaceutically acceptable derivative could be provided in a composition that protects it from the acidic environment of the stomach. For example, the composition can be formulated in an enteric coating that maintains its integrity in the stomach and releases the modulator in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The FXR modulator or pharmaceutically acceptable derivative can also be administered as a component of an elixir, suspension, syrup, wafer, sprinkle, chewing gum or the like. A syrup may contain, in addition to the modulators, sucrose as a sweetening agent and certain preservatives, dyes and colorings and flavors.

The FXR modulator or pharmaceutically acceptable derivative can also be mixed with other agents which do not impair the desired action, or with materials that supplement the desired action, such as antacids, H2 blockers, and diuretics.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents may be used in any of the above dosage forms.

Solvents, include by way of example and without limitation, glycerin, sorbitol, ethyl alcohol and syrup. Examples of preservatives include without limitation glycerin, methyl and propylparaben, benzoic add, sodium benzoate and alcohol. Non-aqueous liquids utilized in emulsions, include by way of example and without limitation, mineral oil and cottonseed oil. Emulsifying agents, include by way of example and without limitation, gelatin, acacia, tragacanth, bentonite, and surfactants such as polyoxyethylene sorbitan monooleate. Suspending agents include, by way of example and without limitation, sodium carboxymethylcellulose, pectin, tragacanth, Veegum and acacia. Diluents include, by way of example and without limitation, lactose and sucrose. Sweetening agents include, by way of example and without limitation, sucrose, syrups, glycerin and artificial sweetening agents such as saccharin. Wetting agents, include by way of example and without limitation, propylene glycol monostearate, sorbitan monooleate, diethylene glycol monolaurate, and polyoxyethylene lauryl ether. Organic acids include, by way of example and without limitation, citric and tartaric acid. Sources of carbon dioxide include, by way of example and without limitation, sodium bicarbonate and sodium carbonate. Coloring agents include, by way of example and without limitation, any of the approved certified water soluble FD and C dyes, and mixtures thereof. Flavoring agents include, by way of example and without limitation, natural flavors extracted from plants such fruits, and synthetic blends of agents which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the modulator or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a agent provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

Tablets and capsules formulations may be coated as known by those of skill in the art in order to modify or sustain dissolution of the FXR modulator or pharmaceutically acceptable derivative. Thus, for example and without limitation, they may be coated with a conventional enterically digestible coating, such as phenylsalicylate, waxes and cellulose acetate phthalate.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients, include by way of example and without limitation, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a FXR modulator or pharmaceutically acceptable derivative is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The FXR modulator or pharmaceutically acceptable derivative diffuses through the outer polymeric membrane in a release rate controlling step. The percentage of FXR modulator or pharmaceutically acceptable derivative contained in such parenteral compositions is highly dependent on the specific nature thereof, as well as the activity of the FXR modulator or pharmaceutically acceptable derivative and the needs of the subject.

Parenteral administration of the FXR modulators or pharmaceutically acceptable derivatives includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Aqueous vehicles include, by way of example and without limitation, Sodium Chloride Injection, Ringers Injection, Isotonic Dextrose Injection, Sterile Water Injection, Dextrose and Lactated Ringers Injection. Nonaqueous parenteral vehicles include, by way of example and without limitation, fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include, by way of example and without limitation, sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include, by way of example and without limitation, ethyl alcohol, polyethylene glycol and propylene glycol for water miscible vehicles and sodium hydroxide, hydrochloric acid, citric acid or lactic acid for pH adjustment.

The concentration of the FXR modulator or pharmaceutically acceptable derivative is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. Preparations for parenteral administration should be sterile, as is known and practiced in the art.

Illustratively, intravenous or intraarterial infusion of a sterile aqueous solution containing a FXR modulator or pharmaceutically acceptable derivative is an effective mode of administration. Another embodiment is a sterile aqueous or oily solution or suspension containing a FXR modulator or pharmaceutically acceptable derivative injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the FXR modulator or pharmaceutically acceptable derivative to the treated tissue(s). The FXR modulator or pharmaceutically acceptable derivative may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The FXR modulator or pharmaceutically acceptable derivative may be suspended in micronized or other suitable form or may be derivatized, e.g., to produce a more soluble active product or to produce a prodrug or other pharmaceutically acceptable derivative. The form of the resulting mixture depends upon a number of factors, including the intended mode of administration and the solubility of the agent in the selected carrier or vehicle. The effective concentration is sufficient for ameliorating the symptoms of the condition and may be empirically determined.

Lyophilized powders can be reconstituted for administration as solutions, emulsions, and other mixtures or formulated as solids or gels.

The sterile, lyophilized powder is prepared by dissolving a FXR modulator or pharmaceutically acceptable derivative provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain, by way of example and without limitation, a single dosage (10-1000 mg, such as 100-500 mg) or multiple dosages of the agent. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, such as about 5-35 mg, for example, about 9-30 mg of lyophilized powder, is added per mL of sterile water or other suitable carrier. The precise amount depends upon the selected agent. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The FXR modulators or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, by way of example and without limitation, have diameters of less than about 50 microns, such as less than about 10 microns.

The FXR modulators or pharmaceutically acceptable derivatives may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the FXR modulator or pharmaceutically acceptable derivative alone or in combination with other pharmaceutically acceptable excipients can also be administered.

These solutions, particularly those intended for ophthalmic use, may be formulated, by way of example and without limitation, as about 0.01% to about 10% isotonic solutions, pH about 5-7, with appropriate salts.

Other routes of administration, such as transdermal patches, and rectal administration are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

Pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is, by way of example and without limitation, about 2 to 3 gm.

Tablets and capsules for rectal administration are manufactured using the same pharmaceutically acceptable substance and by the same methods as for formulations for oral administration.

The FXR modulators or pharmaceutically acceptable derivatives thereof, may also be formulated to be targeted to a particular tissue, receptor, or other area of the body of the subject to be treated. Many such targeting methods are well known to those of skill in the art. Such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a agent provided herein in phosphate buffered saline lacking divalent cations (PBS) is added and the flask shaken until the lipid film is dispersed. The resulting vesicles are washed to remove unencapsulated agent, pelleted by centrifugation, and then resuspended in PBS.

The FXR modulators or pharmaceutically acceptable derivatives for use in the methods may be packaged as articles of manufacture containing packaging material, a FXR modulator or pharmaceutically acceptable derivative thereof provided herein, which is effective for modulating the activity of a farnesoid X receptor or for treatment, of one or more symptoms of nonalcoholic fatty liver disease or cholesterol gallstone disease within the packaging material, and a label that indicates that the FXR modulator or composition, or pharmaceutically acceptable derivative thereof, is used for modulating the activity of farnesoid X receptor for treatment of one or more symptoms of at least one of nonalcoholic fatty liver disease and cholesterol gallstone disease.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

Standard physiological, pharmacological and biochemical procedures are available for testing agents to identify those that possess biological activities that modulate the activity of the farnesoid X receptor. Such assays include, for example, biochemical assays such as binding assays, fluorescence polarization assays, FRET based coactivator recruitment assays (see generally Glickman et al., J. Biomolecular Screening, 7 No. 1 3-10 (2002)), as well as cell based assays including the co-transfection assay, the use of LBD-Gal 4 chimeras, protein-protein interaction assays (see, Lehmann. et al., J. Biol. Chem., 272 (6) 3137-3140 (1997), and gene expression assays.

High throughput screening systems are commercially available (see, e.g., Zymark Corp., Hopkinton, Mass.; Air Technical Industries, Mentor, Ohio; Beckman Instruments Inc., Fullerton, Calif.; Precision Systems, Inc., Natick, Mass.) that enable these assays to be run in a high throughput mode. These systems typically automate entire procedures, including sample and reagent pipetting, liquid dispensing timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Assays that do not require washing or liquid separation steps can be used for high throughput screening systems and include biochemical assays such as fluorescence polarization assays (see for example, Owicki, J., Biomol Screen 2000 October; 5 (5):297) scintillation proximity assays (SPA) (see for example, Carpenter et al., Methods Mol Biol 2002; 190:31-49) and fluorescence resonance energy transfer energy transfer (FRET) or time resolved FRET based coactivator recruitment assays (Mukherjee et al., J Steroid Biochem Mol Biol 2002 July; 81 (3):217-25; (Zhou et al., Mol. Endocrinol. 1998 October; 12 (10):1594-604). Generally such assays can be preformed using either the full length receptor, or isolated ligand binding domain (LBD). In the case of the farnesoid X receptor, the LBD comprises amino acids 244 to 472 of the full length sequence.

If a fluorescently labeled ligand is available, fluorescence polarization assays provide a way of detecting binding of agents to the farnesoid X receptor by measuring changes in fluorescence polarization that occur as a result of the displacement of a trace amount of the label ligand by the agent. Additionally this approach can also be used to monitor the ligand dependent association of a fluorescently labeled coactivator peptide to the farnesoid X receptor to detect ligand binding to the farnesoid X receptor.

The ability of an agent to bind to a receptor, or heterodimer complex with RXR, can also be measured in a homogeneous assay format by assessing the degree to which the agent can compete off a radiolabelled ligand with known affinity for the receptor using a scintillation proximity assay (SPA). In this approach, the radioactivity emitted by a radiolabelled agent generates an optical signal when it is brought into close proximity to a scintillant such as a Ysi-copper containing bead, to which the farnesoid X receptor is bound. If the radiolabelled agent is displaced from the farnesoid X receptor the amount of light emitted from the farnesoid X receptor bound scintillant decreases, and this can be readily detected using standard microplate liquid scintillation plate readers such as, for example, a Wallac MicroBeta reader.

The heterodimerization of the farnesoid X receptor with RXRα can also be measured by fluorescence resonance energy transfer (FRET), or time resolved FRET, to monitor the ability of the agents provided herein to bind to the farnesoid X receptor or other nuclear receptors. Both approaches rely upon the fact that energy transfer from a donor molecule to an acceptor molecule only occurs when donor and acceptor are in close proximity. Typically the purified LBD of the farnesoid X receptor is labeled with biotin then mixed with stoichiometric amounts of europium labeled streptavidin (Wallac Inc.), and the purified LBD of RXRα is labeled with a suitable fluorophore such as CY5™. Equimolar amounts of each modified LBD are mixed together and allowed to equilibrate for at least 1 hour prior to addition to either variable or constant concentrations of the sample for which the affinity is to be determined. After equilibration, the time-resolved fluorescent signal is quantitated using a fluorescent plate reader. The affinity of the agent can then be estimated from a plot of fluorescence versus concentration of agent added.

This approach can also be exploited to measure the ligand dependent interaction of a co-activator peptide with a farnesoid X receptor in order to characterize the agonist or antagonist activity of the agents disclosed herein. Typically the assay in this case involves the use a recombinant Glutathione-S-transferase (GST)-farnesoid X receptor ligand binding domain (LBD) fusion protein and a synthetic biotinylated peptide sequenced derived from the receptor interacting domain of a co-activator peptide such as the steroid receptor coactivator 1 (SRC-1). Typically GST-LBD is labeled with a europium chelate (donor) via a europium-tagged anti-GST antibody, and the coactivator peptide is labeled with allophycocyanin via a streptavidin-biotin linkage.

In the presence of an agonist for the farnesoid X receptor, the peptide is recruited to the GST-LBD bringing europium and allophycocyanin into close proximity to enable energy transfer from the europium chelate to the allophycocyanin. Upon excitation of the complex with light at 340 nm excitation energy absorbed by the europium chelate is transmitted to the allophycocyanin moiety resulting in emission at 665 nm. If the europium chelate is not brought into close proximity to the allophycocyanin moiety there is little or no energy transfer and excitation of the europium chelate results in emission at 615 nm. Thus the intensity of light emitted at 665 nm gives an indication of the strength of the protein-protein interaction. The activity of a farnesoid X receptor antagonist can be measured by determining the ability of a agent to competitively inhibit (i.e., IC₅₀) the activity of an agonist for the farnesoid X receptor.

DNA binding assays can be used to evaluate the ability of an agent to modulate farnesoid X receptor activity. These assays measure the ability of nuclear receptor proteins, including farnesoid X receptor and RXR, to bind to regulatory elements of genes known to be modulated by farnesoid X receptor. In general, the assay involves combining a DNA sequence which can interact with the farnesoid X receptors, and the farnesoid X receptor proteins under conditions, such that the amount of binding of the farnesoid X receptor proteins in the presence or absence of the agent can be measured. In the presence of an agonist, farnesoid X receptor heterodimerizes with RXR and the complex binds to the regulatory element. Methods including, but not limited to DNAse footprinting, gel shift assays, and chromatin immunoprecipitation can be used to measure the amount of farnesoid X receptor proteins bound to regulatory elements.

In addition a variety of cell based assay methodologies may be successfully used in screening assays to identify and profile the specificity of agents described herein. These approaches include the co-transfection assay, translocation assays, and gene expression assays.

Three basic variants of the co-transfection assay strategy exist, co-transfection assays using full-length farnesoid X receptor, co-transfection assays using chimeric farnesoid X receptors comprising the ligand binding domain of the farnesoid X receptor fused to a heterologous DNA binding domain, and assays based around the use of the mammalian two hybrid assay system.

The basic co-transfection assay is based on the co-transfection into the cell of an expression plasmid to express the farnesoid X receptor in the cell with a reporter plasmid comprising a reporter gene whose expression is under the control of DNA sequence that is capable of interacting with that nuclear receptor. (See for example U.S. Pat. Nos. 5,071,773; 5,298,429, 6,416,957, WO 00/76523). Treatment of the transfected cells with an agonist for the farnesoid X receptor increases the transcriptional activity of that receptor which is reflected by an increase in expression of the reporter gene, which may be measured by a variety of standard procedures.

For those receptors that function as heterodimers with RXR, such as the farnesoid X receptor, the co-transfection assay typically includes the use of expression plasmids for both the farnesoid X receptor and RXR. Typical co-transfection assays require access to the full-length farnesoid X receptor and suitable response elements that provide sufficient screening sensitivity and specificity to the farnesoid X receptor.

Genes encoding the following full-length previously described proteins, which are suitable for use in the co-transfection studies and profiling the agents described herein, include rat farnesoid X receptor (GenBank Accession No. NM_{—}021745), human farnesoid X receptor (GenBank Accession No. NM_{—}005123), human RXR α (GenBank Accession No. NM_{—}002957), human RXR β (GenBank Accession No. XM_{—}042579), human RXR γ (GenBank Accession No. XM_{—}053680),

Reporter plasmids may be constructed using standard molecular biological techniques by placing cDNA encoding for the reporter gene downstream from a suitable minimal promoter. For example luciferase reporter plasmids may be constructed by placing cDNA encoding firefly luciferase immediately down stream from the herpes virus thymidine kinase promoter (located at nucleotides residues −105 to +51 of the thymidine kinase nucleotide sequence) which is linked in turn to the various response elements.

Numerous methods of co-transfecting the expression and reporter plasmids are known to those of skill in the art and may be used for the co-transfection assay to introduce the plasmids into a suitable cell type. Typically such a cell will not endogenously express farnesoid X receptors that interact with the response elements used in the reporter plasmid.

Numerous reporter gene systems are known in the art and include, for example, alkaline phosphatase Berger, J., et al (1988) Gene 66 1-10; Kain, S. R. (1997) Methods. Mol. Biol. 63 49-60), β-galactosidase (See, U.S. Pat. No. 5,070,012, issued Dec., 3, 1991 to Nolan et al., and Bronstein, I., et al., (1989) J. Chemilum. Biolum. 4 99-111), chloramphenicol acetyltransferase (See Gorman et al., Mol Cell Biol. (1982) 2 1044-51), β-glucuronidase, peroxidase, β-lactamase (U.S. Pat. Nos. 5,741,657 and 5,955,604), catalytic antibodies, luciferases (U.S. Pat. Nos. 5,221,623; 5,683,888; 5,674,713; 5,650,289; 5,843,746) and naturally fluorescent proteins (Tsien, R. Y. (1998) Annu. Rev. Biochem. 67 509-44).

The use of chimeras comprising the ligand binding domain (LBD) of the farnesoid X receptor fused to a heterologous DNA binding domain (DBD) expands the versatility of cell based assays by directing activation of the farnesoid X receptor in question to defined DNA binding elements recognized by defined DNA binding domain (see WO95/18380). This assay expands the utility of cell based co-transfection assays in cases where the biological response or screening window using the native DNA binding domain is not satisfactory.

In general the methodology is similar to that used with the basic co-transfection assay, except that a chimeric construct is used in place of the full-length farnesoid X receptor. As with the full-length farnesoid X receptor, treatment of the transfected cells with an agonist for the farnesoid X receptor LBD increases the transcriptional activity of the heterologous DNA binding domain which is reflected by an increase in expression of the reporter gene as described above. Typically for such chimeric constructs, the DNA binding domains from defined farnesoid X receptors, or from yeast or bacterially derived transcriptional regulators such as members of the GAL 4 and Lex A/Umud super families are used.

A third cell based assay of utility for screening agents is a mammalian two-hybrid assay that measures the ability of the nuclear hormone receptor to interact with a cofactor in the presence of a ligand. (See for example, U.S. Pat. Nos. 5,667,973, 5,283,173 and 5,468,614). The basic approach is to create three plasmid constructs that enable the interaction of the farnesoid X receptor with the interacting protein to be coupled to a transcriptional readout within a living cell. The first construct is an expression plasmid for expressing a fusion protein comprising the interacting protein, or a portion of that protein containing the interacting domain, fused to a GAL4 DNA binding domain. The second expression plasmid comprises DNA encoding the farnesoid X receptor fused to a strong transcription activation domain such as VP16, and the third construct comprises the reporter plasmid comprising a reporter gene with a minimal promoter and GAL4 upstream activating sequences.

Once all three plasmids are introduced into a cell, the GAL4 DNA binding domain encoded in the first construct allows for specific binding of the fusion protein to GAL4 sites upstream of a minimal promoter. However because the GAL4 DNA binding domain typically has no strong transcriptional activation properties in isolation, expression of the reporter gene occurs only at a low level. In the presence of a ligand, the farnesoid X receptor-VP16 fusion protein can bind to the GAL4-interacting protein fusion protein bringing the strong transcriptional activator VP16 in close proximity to the GAL4 binding sites and minimal promoter region of the reporter gene. This interaction significantly enhances the transcription of the reporter gene, which can be measured for various reporter genes as described above. Transcription of the reporter gene is thus driven by the interaction of the interacting protein and farnesoid X receptor in a ligand dependent fashion.

An agent can be tested for the ability to induce nuclear localization of a nuclear protein receptor, such as farnesoid X receptor. Upon binding of an agonist, farnesoid X receptor translocates from the cytoplasm to the nucleus. Microscopic techniques can be used to visualize and quantitate the amount of farnesoid X receptor located in the nucleus. In some embodiments, this assay can utilize a chimeric farnesoid X receptor fused to a fluorescent protein.

An agent can also be evaluated for its ability to increase or decrease the expression of genes known to be modulated by the farnesoid X receptor in vivo, using Northern-blot, RT PCR or oligonucleotide microarray analysis to analyze RNA levels. Western-blot analysis can be used to measure expression of proteins encoded by farnesoid X receptor target genes. Genes known to be regulated by the farnesoid X receptor include cholesterol 7α-hydroxylase (CYP7A1), the rate limiting enzyme in the conversion of cholesterol to bile acids, fatty acid synthase (FAS), the small heterodimer partner (SHP), the bile salt export pump (BSEP, ABCB11), canalicular bile acid export protein, the multiple drug resistance-2 (MDR-2, ABCB4), sodium taurocholate cotransporting polypeptide (NTCP, SLC10A1) and intestinal bile acid binding protein (I-BABP).

Expression of a farnesoid X receptor target gene can be conveniently normalized to an internal control and the data plotted as fold induction relative to untreated or vehicle treated cells. A control agent, such as an agonist, may be included along with DMSO as high and low controls respectively for normalization of the assay data.

Any agent which is a candidate for modulation of the farnesoid X receptor may be tested by the methods described above. Generally, though not necessarily, agents are tested at several different concentrations and administered one or more times to optimize the chances that activation of the receptor will be detected and recognized if present. Typically assays are performed in triplicate, for example, and vary within experimental error by less than about 15%. Each experiment is typically repeated about three or more times with similar results.

In some embodiments, the effects of agents and compositions on farnesoid X receptor gene expression and activity can be evaluated in vivo. After the administration of agents to animals, various tissues can be harvested to determine the effect of agents on factors directly or indirectly regulated by farnesoid X receptor. For example and without limitation, factors directly or indirectly regulated by farnesoid X receptor can include FAS, SHP, BSEP, MDR2, VCAM-1, ICAM-1, TNFα, MCP-1, KC, TIMP-1, CYP2E1, CK-18, a-SMA, Col1a2, TGFβ, ALT, and AST. Additional factors directly or indirectly regulated by farnesoid X receptor can include at least one positive acute phase protein and at least one MMP. In some embodiments, the at least one positive acute phase protein is selected from CRP, SAP, and at least one SAA. In some embodiments, the at least one SAA is SAA_{—}3. In some embodiments, the at least one MMP is MMP-2, MMP-9, and MMP-14. In some embodiments, the levels of mRNA can be measured with Northern blot, RT-PCR, or oligonucleotide microarray analysis. In some embodiments, protein levels can be measured with Western blot or Enzyme linked immunosorbent assay (ELISA).

In some embodiments, the activities of the factors are measured. An elevated level of at least one of ALT and AST may be used to diagnose and monitor liver disease. AST is normally expressed within liver, cardiac, skeletal muscle cells, and other tissues. In some embodiments, analysis of the level of AST is measured in combination with analysis of the level of ALT to monitor liver damage. In some embodiments, the level of ALT activity in serum is monitored using enzymatic assays that measure the conversion of alanine to α-ketoglutaric acid. In some embodiments, the serum level of AST is monitored using assays that measure the conversion of aspartate to α-ketoglutaric acid. In some embodiments, the serum level of CK-18 is used to diagnose liver disease including but not limited to nonalcoholic steatohepatitis (NASH). In some embodiments, the amount of full length CK-18 is measured. In some embodiments, at least one proteolytically cleaved form of CK-18 is measured.

Provided herein are methods for identifying a FXR modulator in vivo. Feeding animals, for example but not limited to mice and rats, with a lithogenic diet high in cholesterol and cholic acid, can induce at least one feature or symptom of nonalcoholic fatty liver disease or cholesterol gallstone disease. In some embodiments, a FXR modulator modulates at least one feature of nonalcoholic fatty liver disease selected from, for example and without limitation, neutral lipid deposition, intracellular lipid droplet formation, Kupffer cell activation, inflammatory cell infiltration, inflammatory cholangitis, portal inflammation, fibrosis, oxidative stress, and acute phase response in the liver. In some embodiments, a FXR modulator modulates one of gallstone incidence, gallstone dissolution time, bile cholesterol levels, bile salt/phospholipid ratios, biliary symptoms, and gallbladder inflammation.

Provided herein are methods involving both in vitro and in vivo uses of an agent that modulates farnesoid X receptor activity. Provided are methods of treating at least one of nonalcoholic fatty liver disease and cholesterol gallstone disease with an agent that modulates farnesoid X receptor activity. Such agents will typically exhibit farnesoid X receptor agonist, partial agonist, partial antagonist or antagonist activity in one of the in vitro or in vivo assays described herein. Methods of altering farnesoid X receptor activity, by contacting the receptor with at least one agent, are provided.

Treatment with a FXR modulator may be associated with side effects. Provided herein is method of treating at least one of nonalcoholic fatty liver disease and cholesterol gallstone disease with an agent selected to have fewer side effects based on its profile and activities in assays testing for farnesoid X receptor activity. For example, an agent may be selected for high activity in treating features of at least one of nonalcoholic fatty liver disease and cholesterol gallstone disease and low activity in assays that do not monitor features of at least one of nonalcoholic fatty liver disease and cholesterol gallstone disease.

Administering at least one FXR modulator or pharmaceutically acceptable derivative can potentiate the effects of known agents useful for the treatment of cholesterol gallstone disease. Contemplated herein is combination therapy using at least one FXR modulator or a pharmaceutically acceptable derivative thereof, in combination with at least other agent selected from chenodeoxycholic acid, ursodeoxycholic acid and any prescribed drug for the targeted indication.

The FXR modulator or pharmaceutically acceptable derivative thereof, is administered simultaneously with, prior to, or after administration of one or more of the above agents.

The invention is further illustrated by the following non-limiting examples.

### EXAMPLES

### Example 1

Male C57BL/6 mice were fed a standard chow diet or a Paigen diet (TestDiet 32350 containing 7.5% Cocoa Butter, 1.25% cholesterol, and 0.5% cholic acid). Paigen diet fed mice were administered vehicle or 30 mg/kg FXR agonist, Compound A (isopropyl 3-(3,4-difluorobenzoyl)-1,1-dimethyl-1,2,3,6-tetrahydroazepino[4,5-b]indole-5-carboxylate) orally once a day for 4 weeks. Three hours after the final treatment, mice were euthanized and blood and liver were collected for analyses.

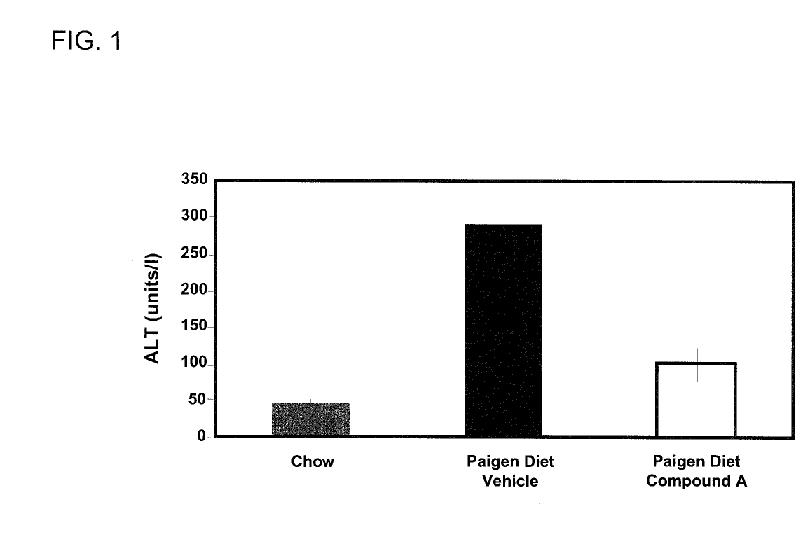

FIG. 1 shows the serum alanine aminotransferase (ALT) activity level in mice fed a standard chow diet (n=5), vehicle treated mice fed a Paigen diet (n=19), and Compound A treated mice fed a Paigen diet (n=19). Data are presented as the mean±standard error (SE). An elevated level of ALT activity was observed in the serum of mice fed a Paigen diet compared to mice fed a standard chow diet. Compound A treatment preserved liver function as shown by the significant reduction in the level of ALT activity from 292.1±33.6 U/L in vehicle treated Paigen diet fed mice to 98.1±22.5 U/L in Compound A treated Paigen diet fed mice (p<0.0001).

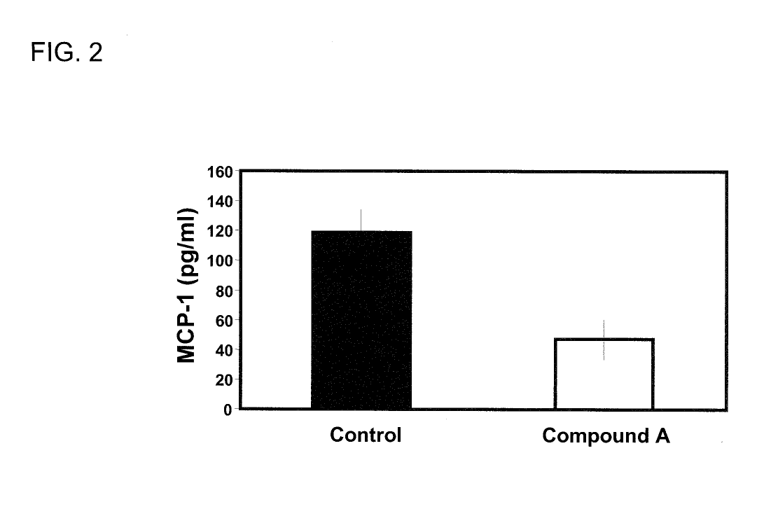

FIG. 2 shows the serum level of monocyte chemotactic protein-1 (MCP-1) in vehicle treated mice fed a Paigen diet, and Compound A treated mice fed a Paigen diet. Data are presented as the mean±SE. 6 mice were tested in each group. Compound A treatment significantly reduced the level of MCP-1 (p<0.0001). The reduction in the level of MCP-1 may contribute to the reduction in inflammatory cell infiltration observed in Compound A treated mice.

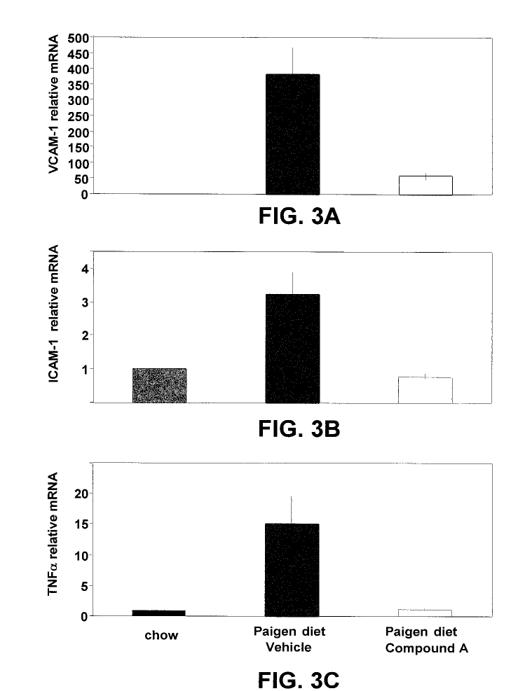

Real-time RT PCR analysis of liver RNA was performed. FIGS. 3A, 3B, and 3C show the expression levels of vascular cell adhesion molecule 1 (VCAM-1), intercellular adhesion molecule-1 (ICAM-1), and tumor necrosis factor α (TNFα). Expression levels were normalized to GAPDH, and the mean level of expression of each gene in mice fed a standard chow diet was defined as 1.0. 6 mice were tested in each group. Data are presented as the mean±SE. Compound A treatment significantly inhibited Paigen diet induction of VCAM-1, ICAM-1 and TNFα expression levels (p<0.0001).

### Example 2

Male C57BL/6 mice were fed a chow diet or a Paigen diet. Paigen fed mice were administered vehicle or 30 mg/kg Compound A orally once a day for 4 weeks. Three hours after the final treatment, mice were euthanized, and histological analysis of livers was performed. Frozen liver sections (5 μm) were prepared and stained with Oil Red O, hematoxylin and eosin (H&E), and Masson's trichrome (Trichrome).

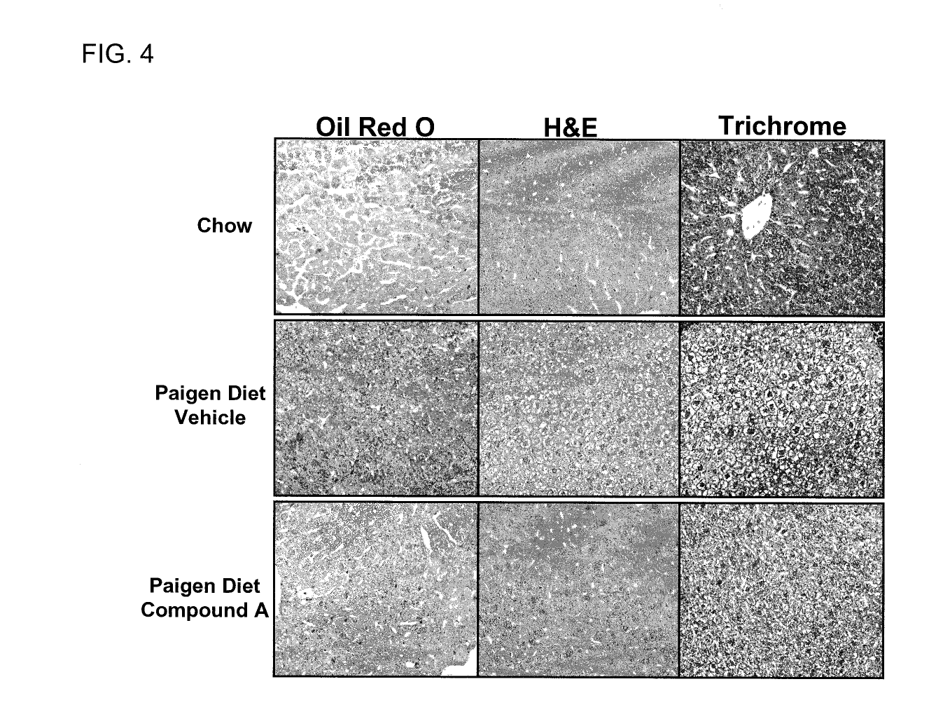

FIG. 4 shows representative liver sections from mice fed a standard chow diet, vehicle treated mice fed a Paigen diet, and Compound A treated mice fed a Paigen diet. Oil Red O staining revealed significantly less red content in liver sections from Compound A compared with vehicle treated Paigen fed mice indicating that Compound A treatment caused a reduction in neutral lipid accumulation in the liver. H&E and Trichrome staining revealed the presence of intracellular vacuoles characteristic of lipid droplets in the livers of mice fed a Paigen diet. Treatment with Compound A reduced the number and severity of the vacuoles induced by a Paigen diet.

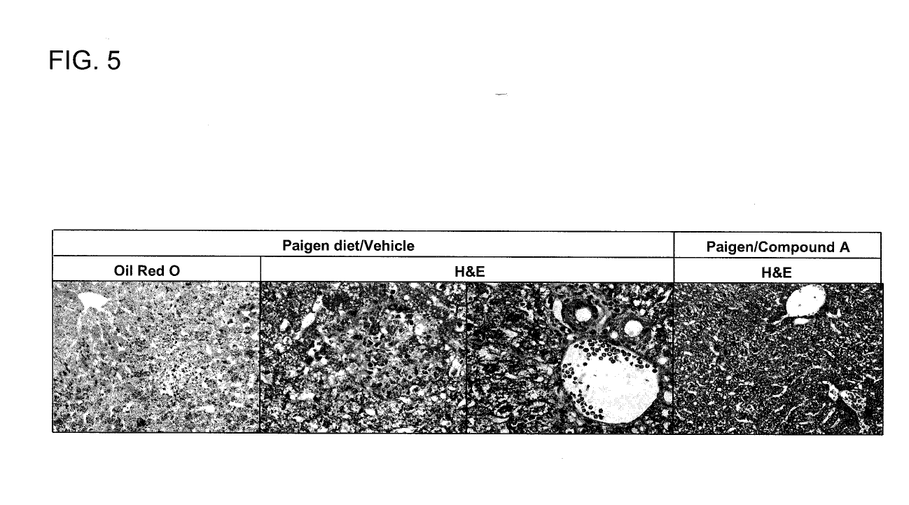

FIG. 5 shows that inflammatory cholangitis with portal inflammation was significantly more severe in the livers of vehicle treated compared to Compound A treated mice fed a Paigen diet.

Histological analyses of livers shown in FIGS. 4 and 5 indicate that Compound A treatment attenuated Paigen diet induced liver damage.

### Example 3

Male C57BL/6 mice were fed a chow diet or a Paigen diet. Paigen fed mice were administered vehicle or 30 mg/kg Compound A orally once a day for 4 weeks. Three hours after the final treatment, mice were euthanized, and real-time RT PCR analysis of liver RNA was performed.

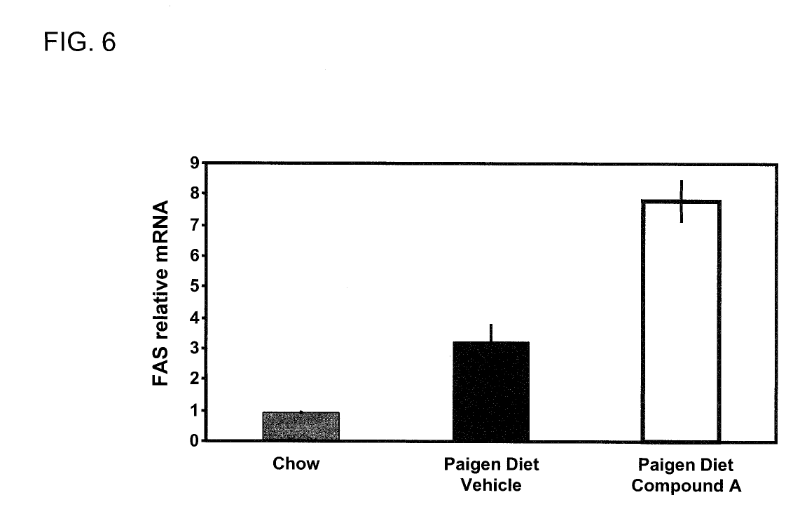

FIG. 6 shows the expression level of fatty acid synthase (FAS). Real-time RT PCR analysis of liver RNA was performed. The expression level of FAS was normalized to GAPDH, and the mean level of expression of FAS in mice fed a standard chow diet was defined as 1.0. 6 mice were tested in each group. Data are presented as the mean±SE. The expression level of FAS were induced by the Paigen diet. Compound A treatment further induced FAS expression by 2.4 fold (p<0.0001). Elevated FAS expression may compensate for the reduced neutral lipid in Compound A treated mice compared with vehicle treated mice fed a Paigen diet.

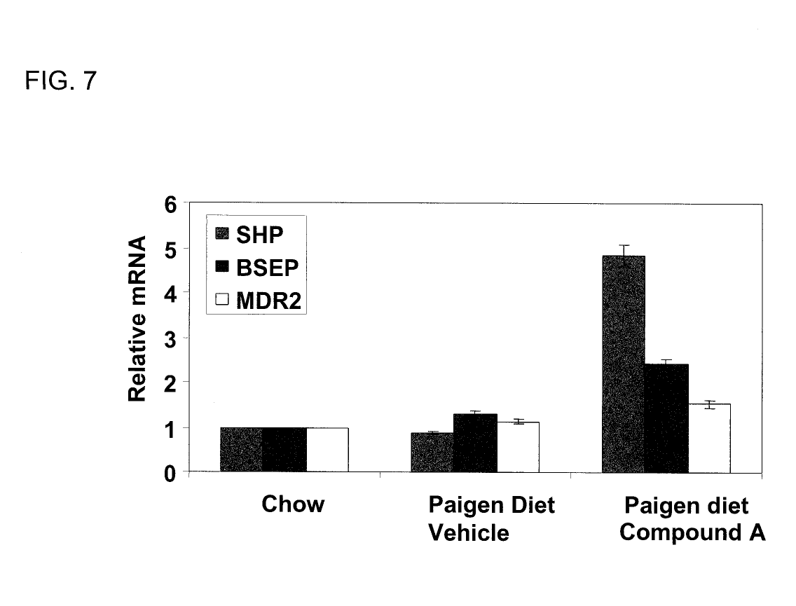

As shown in FIG. 7, Compound A treatment also increased liver expression levels of small heterodimer partner (SHP) by 4.85 fold, bile salt export pump (BSEP) which is involved in bile acid secretion by 2.41 fold, and multiple drug resistance-2 (MDR2) which is involved in phospholipid secretion by 1.4 fold.

### Example 4

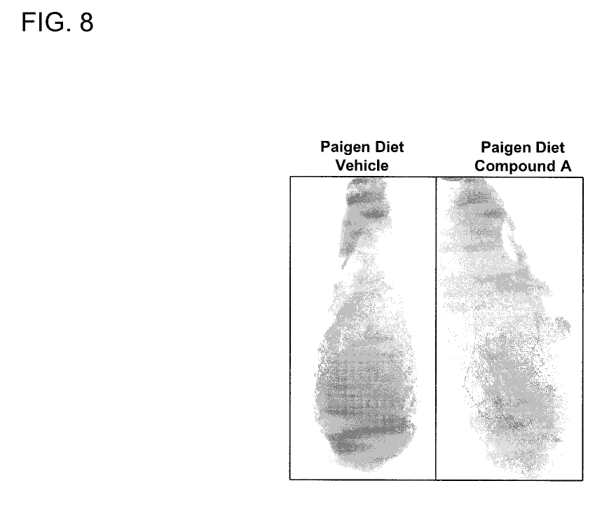

To study gallstone formation, gallbladders were harvested from mice fed a Paigen diet and administered vehicle or 30 mg/kg Compound A orally once a day for 4 weeks. FIG. 8 shows representative images of the gross morphology of gallbladders harvested from the mice. Cholesterol gallstones were clearly visible in the gallbladders of vehicle but not Compound A treated mice.

Table 1 shows the incidence of gallstone formation in the mice. As shown in the column labeled gallstone formation, Compound A treatment reduced the incidence of gallstone formation from 14/19 (73.7%) in vehicle treated mice to 0/20 (0%) in Compound A treated mice.

TABLE 1

| | Gallstone formation | Gallstone dissolution |
|---|---|---|
| Vehicle | 14/19 | 13/26 |
| Compound A | 0/20 | 5/20 |

To study gallstone dissolution, gallbladders were harvested from mice treated as follows. C57BL/6 mice were fed a Paigen diet for 4 weeks to initiate gallstone formation. Subsequently these mice were administered vehicle or 30 mg/kg Compound A orally once a day and fed a standard chow diet for 4 weeks to study the effect of Compound A on preformed gallstones.

Table 1 also shows the incidence of gallstones in mice with preformed gallstones. As shown in the column labeled gallstone dissolution, Compound A treatment caused significant gallstone dissolution with a decrease of gallstone incidence from 13/26 (50%) in vehicle treated mice to 5/20 (25%) in Compound A treated mice.

### Example 5

Disrupted cholesterol, phospholipid, and bile salt homeostasis contributes to the formation of gallstones. Gallbladder bile lipid composition was quantitated from mice treated as follows. A group of mice (CHOW) was fed a standard chow diet. Another group of mice was fed a Paigen diet and administered vehicle (Paigen/Vehicle) or 30 mg/kg Compound A (Paigen/Compound A) orally once a day for 4 weeks. To preform gallstones, mice were fed initially a Paigen diet for 4 weeks and subsequently fed a standard chow diet and administered vehicle (Preformed/Vehicle) or 30 mg/kg Compound A (Preformed/Compound A) orally once a day for 4 weeks. Table 2 shows the number of mice (N) and the amounts of cholesterol (CE), phospholipids (PL), and bile salt (BS) measured in each group. Data are presented as the mean±SE.

TABLE 2

| | N | CE(mM) | PL(mM) | BS(mM) |
|---|---|---|---|---|
| Chow | 4 | 2.69 ± 0.14 | 14.93 ± 1.75 | 111.59 ± 23.55 |
| Paigen/Vehicle | 6 | 9.99 ± 0.79 | 26.62 ± 2.35 | 111.60 ± 13.03 |
| Paigen/Compound A | 8 | 7.70 ± 0.28 | 20.19 ± 1.00 | 110.42 ± 24.79 |
| Preformed/Vehicle | 10 | 3.74 ± 0.31 | 19.97 ± 1.92 | 154.57 ± 17.58 |
| Preformed/ | 8 | 7.54 ± 0.86 | 27.54 ± 2.14 | 120.06 ± 14.28 |
| Compound A | | | | |

Mice fed a Paigen diet had significantly elevated levels of bile cholesterol and phospholipids. There was no significant difference in bile salt levels in Paigen diet fed mice compared to mice fed a standard chow diet.

The lower cholesterol and phospholipid levels in Compound A treated mice (Paigen/Compound A) compared to vehicle treated mice fed a Paigen diet (Paigen/Vehicle) indicate that Compound A treatment attenuated the increases in cholesterol and phospholipid levels induced by the Paigen diet. Reduction in bile cholesterol levels by Compound A treatment may contribute to gallstone treatment.

Lower levels of bile salt and higher levels of cholesterol and phospholipids were observed in mice with preformed gallstones treated with Compound A (Preformed/Compound A) compared to those treated with vehicle (Preformed/Vehicle). The bile salt/phospholipid ratio was significantly decreased in mice treated with Compound A compared to vehicle treated mice. Compound A administration may contribute to gallstone treatment because cholesterol crystal precipitation occurs at a slower rate in the presence of excess phospholipids. A shift in the bile acid pool induced by Compound A administration may also contribute to gallstone dissolution.

### Example 6

Male C57BL/6 mice were fed a standard chow diet or a methionine/choline deficient (MCD) diet (MCD diet; TD 90262, Harlan Teklad). MCD diet fed mice develop fibrosis and NAFLD including steatosis and NASH due to impaired mitochondrial β-oxidation and reduced hepatic triglyceride secretion.

Mice were administered vehicle or 30 mg/kg Compound A orally once a day for 4 weeks. Three hours after the final treatment, mice were euthanized, and blood and liver were collected for analyses.

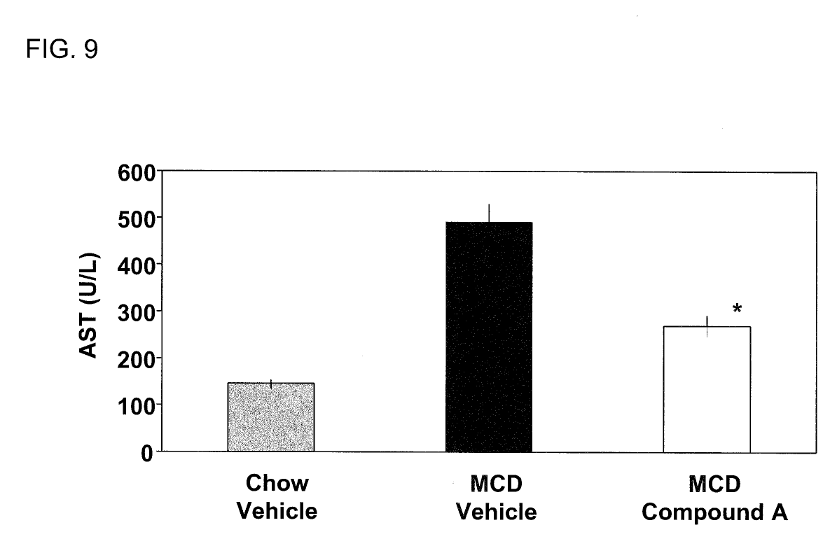

FIG. 9 shows the serum level of aspartate aminotransferase (AST) activity in vehicle treated mice fed a standard chow diet (n=6), vehicle treated mice fed a MCD diet (n=11), and Compound A treated mice fed a MCD diet (n=12). Data are presented as the mean±standard error (SE). Elevated AST activity levels were observed in the serum of mice fed a MCD diet compared to mice fed a standard chow diet. Compound A treatment preserved liver function by significantly reducing AST activity levels from 491±38.9 U/L in vehicle treated MCD diet fed mice to 268±77 U/L in Compound A treated MCD diet fed mice (p<0.0001, indicated by *).

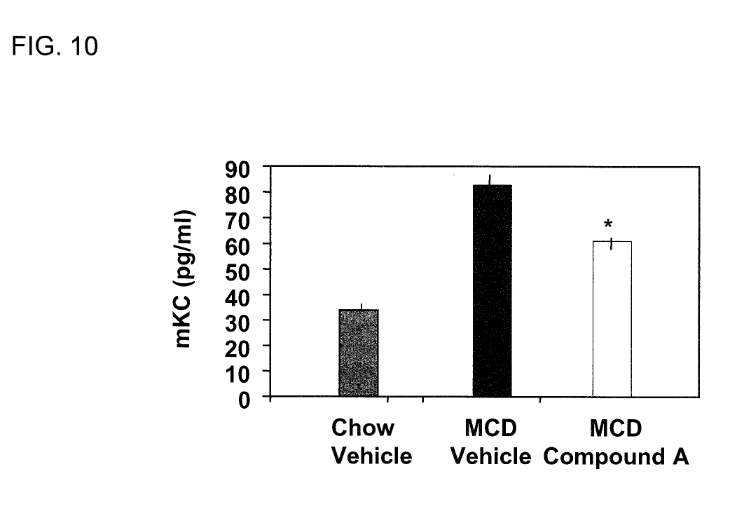

Shown in FIG. 10 is the serum level of mouse keratinocyte-derived chemokine (mKC) in vehicle treated mice fed a standard chow diet (n=6), vehicle treated mice fed a MCD diet (n=11), and Compound A treated mice fed a MCD diet (n=12). Data are presented as the mean±SE. Compound A treatment lowered inflammation as evidenced by reduced mKC levels in Compound A MCD fed mice compared to vehicle treated MCD fed mice (p<0.05, indicated by *).

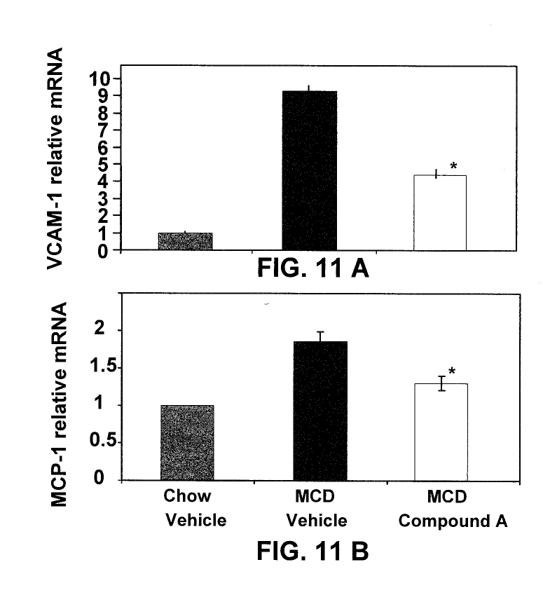

FIGS. 11A and 11B show the expression levels of VCAM-1 and MCP-1, respectively. Real-time RT PCR analysis of liver RNA was performed. Expression levels were normalized to GAPDH, and the mean level of expression of each gene in vehicle treated mice fed a standard chow diet was defined as 1.0. 7 mice were tested in each group. Data are presented as the mean±SE. Compound A treatment significantly inhibited expression of the inflammatory mediators, VCAM-1 and MCP-1 (p<0.001, indicated by *).

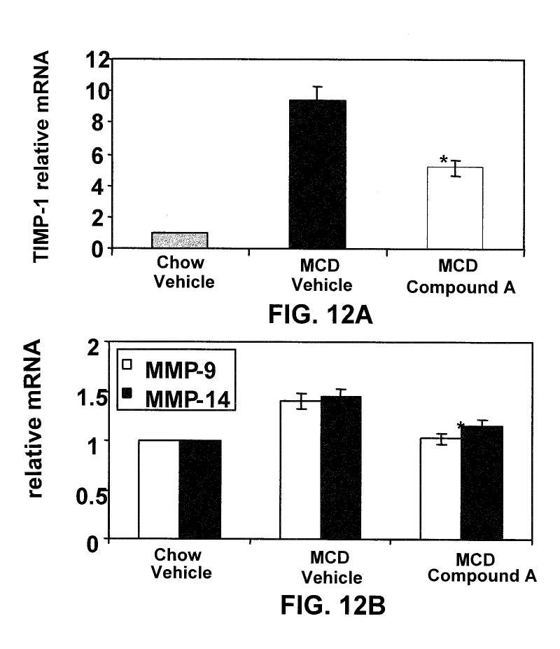

FIGS. 12A and 12B show the liver expression levels of tissue inhibitor of metalloproteinase 1 (TIMP-1), matrix metalloproteinase-9 (MMP-9), and MMP-14. Expression levels were normalized to GAPDH, and the mean level of expression of each gene in vehicle treated mice fed a standard chow diet was defined as 1.0. 7 mice were tested in each group. Data are presented as the mean±SE. Compound A treatment reduced liver fibrosis through inhibition of MCD diet induction of TIMP-1, MMP-9, and MMP-14 expression levels (p<0.001, indicated by *).

### Example 7

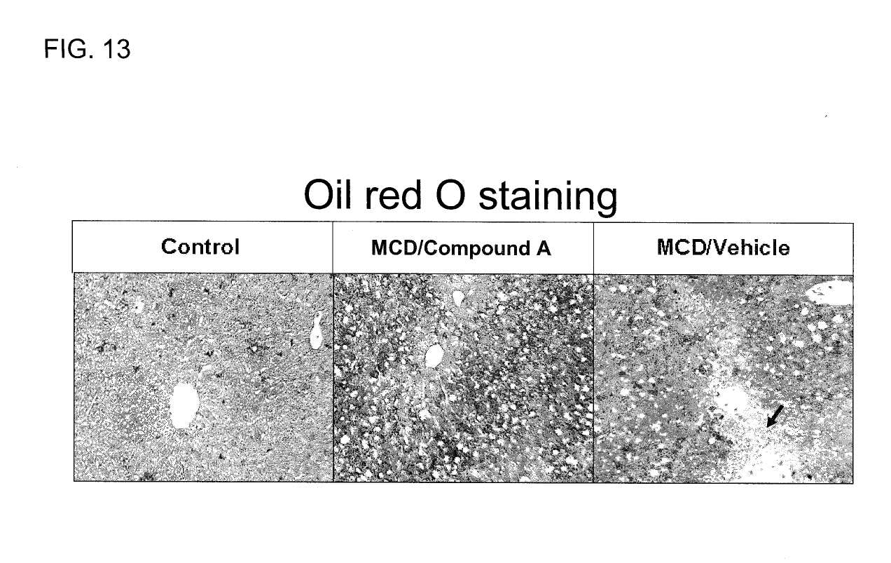

FIGS. 13, 14, and 15 show representative liver sections from mice fed a standard chow diet, vehicle treated mice fed a MCD diet, and Compound A (30 mg/kg orally once a day) treated mice fed a MCD diet for 4 weeks. Oil Red O staining of liver sections shown in FIG. 13 indicated similar red content in liver sections from Compound A compared with vehicle treated MCD fed mice suggesting that Compound A treatment had no effect in the neutral lipid accumulation in the MCD fed mice liver. Inflammatory cell infiltration (labeled with an arrow) was observed in the vehicle treated mice fed a MCD diet.

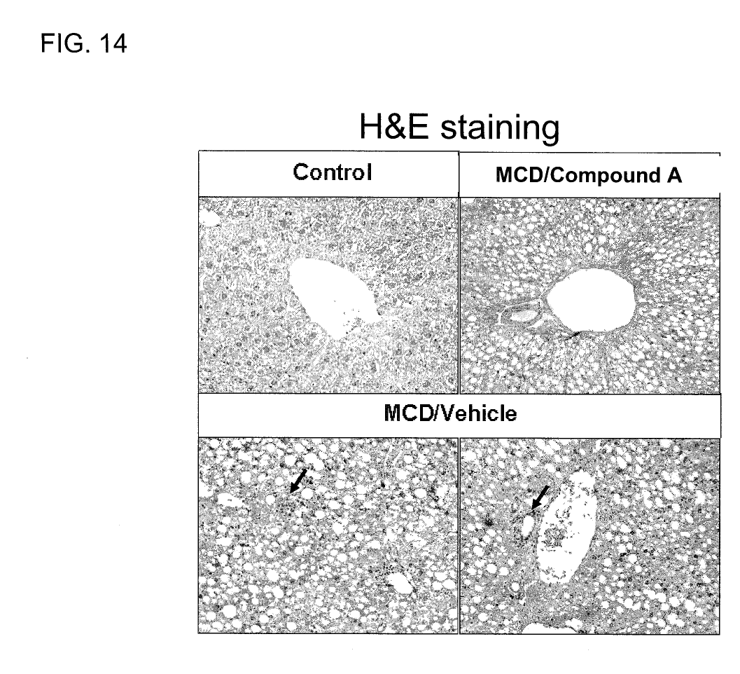

Hemotoxylin and eosin (H&E) staining of sections shown in FIG. 14 indicated that inflammatory cell infiltration and portal inflammation were more severe in the livers of vehicle treated compared to Compound A treated mice fed MCD diet. The arrows in FIG. 14 indicate the presence of inflammatory cells.

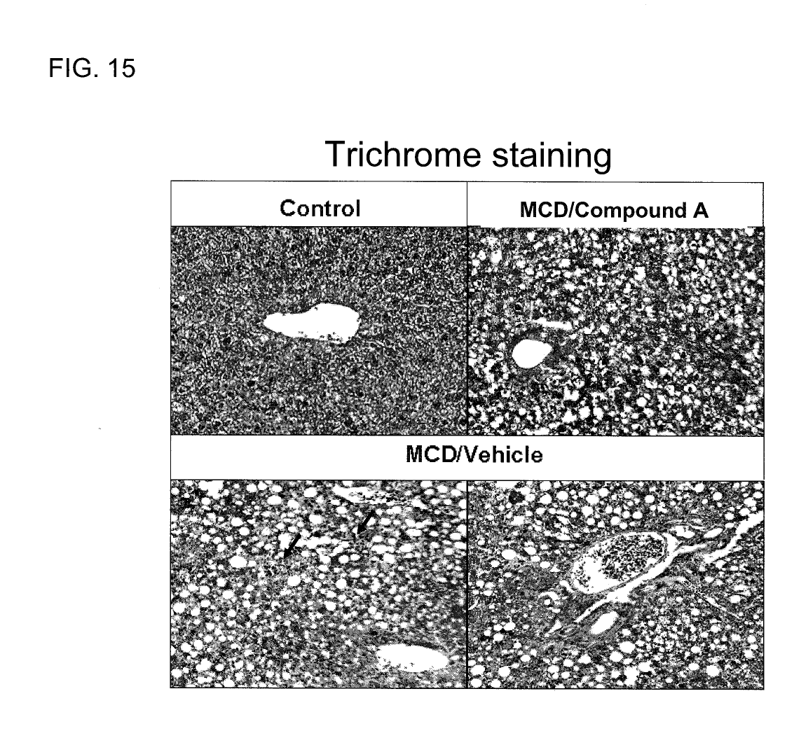

Trichrome staining of liver sections shown in FIG. 15 indicated that inflammatory cell infiltration and fibrosis were more severe in the livers of vehicle treated compared to Compound A treated mice fed MCD diet.

Histological analyses of liver sections showed that Compound A treatment preserved liver function, and reduced liver inflammation and fibrosis. This liver protection effect of Compound A did not appear to be mediated by neutral lipid accumulation reduction as indicated by the H&E staining in FIG. 14.

### Example 8

Male C57BL/6 mice were fed a standard chow diet or a MCD diet and treated with either vehicle or 30 mg/kg Compound A orally once a day for 4 weeks. Three hours after the final treatment, mice were euthanized and liver expression of CYP2E1, a reactive oxygen species (ROS) generating microsomal enzyme, was analyzed.

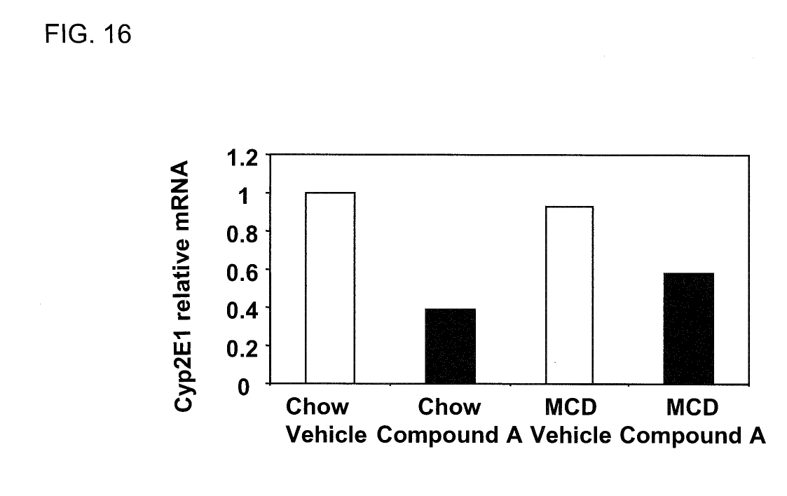

FIG. 16 shows the liver expression level of CYP2E1. Expression levels were normalized to GAPDH, and the mean level of expression of each gene in vehicle treated mice fed a standard chow diet was defined as 1.0. 7 mice were tested in each group. Data are presented as the mean±SE. Compound A treatment reduced liver oxidative stress as evidenced by significant inhibition of CYP2E1 expression levels independent of MCD diet treatment (p<0.001).

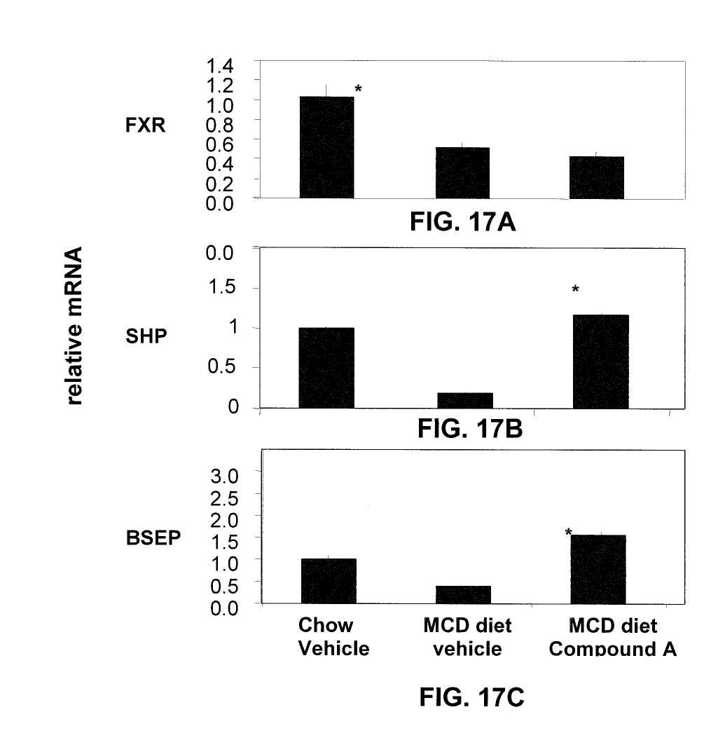

FIGS. 17A, 17B, and 17C show the liver expression levels of FXR and its target genes small heterodimer partner (SHP) and bile salt export pump (BSEP). The expression level of FXR was greatly suppressed by the MCD diet. Compound A treatment normalized FXR target gene expression but did not normalize the level of FXR gene expression.

### Example 9

Mice were fed a standard chow diet or a MCD diet and treated with either vehicle or 30 mg/kg Compound A orally once a day for 4 weeks.

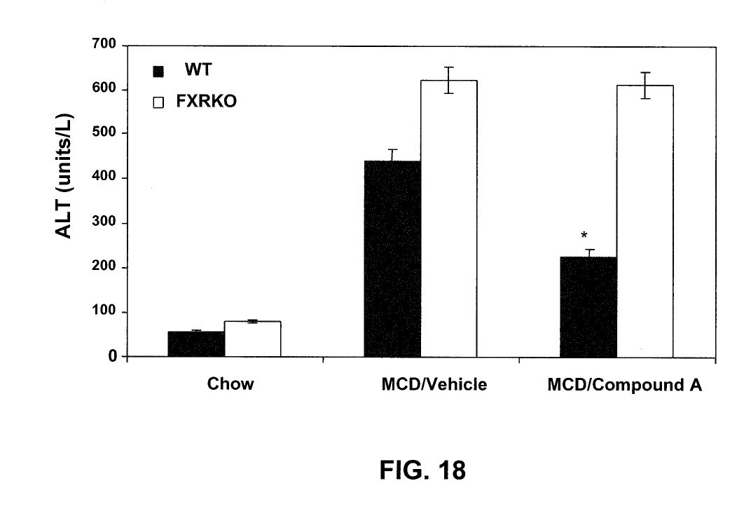

FIG. 18 shows the serum level of ALT activity in wildtype (WT) and FXR deficient (FXRKO) mice fed a standard chow diet, vehicle treated WT and FXRKO mice fed a MCD diet, and Compound A treated WT and FXRKO mice fed a MCD diet. Compound A treatment decreased ALT levels and had liver protective effects in wildtype mice. Furthermore, the hepatic protective effects of Compound A are mediated by FXR. (*P<0.01, WT/MCD/Vehicle vs. WT/MCD/Compound A).

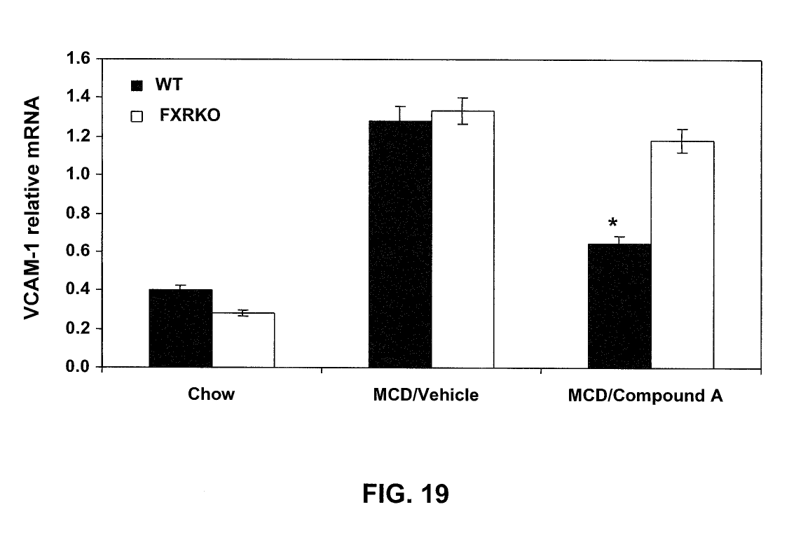

FIG. 19 shows the gene expression level of vascular cell adhesion molecule 1 (VCAM-1) in the livers of WT and FXRKO mice fed a standard chow diet, vehicle treated WT and FXRKO mice fed a MCD diet, and Compound A treated WT and FXRKO mice fed a MCD diet. The effect of Compound A on decreasing VCAM-1 levels is mediated by FXR. (*P<0.001, WT/MCD/Vehicle vs. WT/MCD/Compound A).

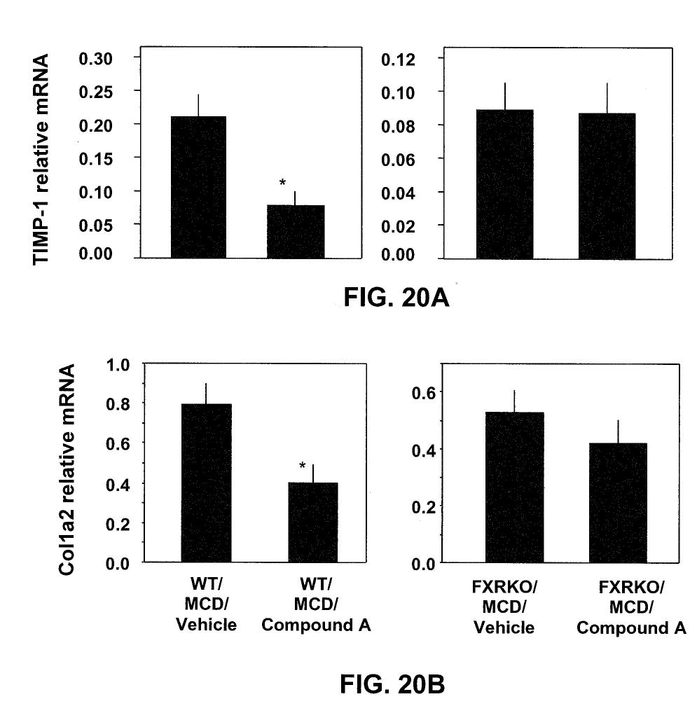

FIG. 20A shows the gene expression level of tissue inhibitor of metalloproteinase-1 (TIMP-1), and FIG. 20B shows the expression level of collagen, type I, alpha 2 (Col1a2), in the livers of WT and FXRKO mice fed a standard chow diet, vehicle treated WT and FXRKO mice fed a MCD diet, and Compound A treated WT and FXRKO mice fed a MCD diet. These data show that the reduction of fibrosis markers including Col1a2 and TIMP-1 by Compound A treatment is mediated by FXR. (FIG. 20A, *P<0.001, WT/MCD/Vehicle vs. WT/MCD/Compound A. FIG. 20B, *P<0.01, WT/MCD/Vehicle vs. WT/MCD/Compound A).

### Example 10

C57Bl/6 mice were fed a MCD diet for 2 weeks to induce the development of NASH to study the effect of Compound A treatment on preformed NASH. These mice were then maintained on the MCD diet while being treated with vehicle or Compound A. Mice were euthanized 2 weeks (2 w), and 4 weeks (4 w) after the beginning of treatment with vehicle or Compound A for analysis. As controls, mice were fed a chow diet or fed a MCD diet for 2 weeks, and then euthanized for analysis.

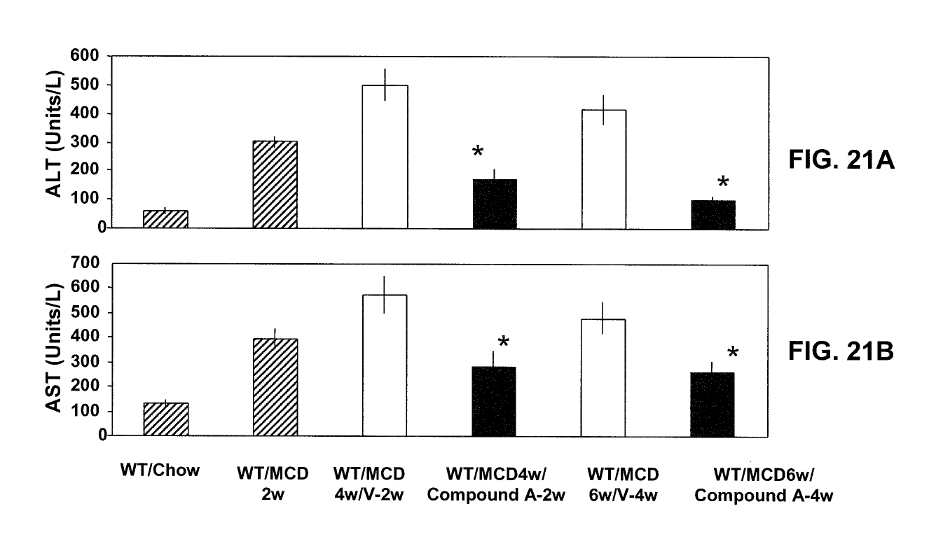

FIG. 21A shows the serum level of ALT activity and FIG. 21B shows the serum level of aspartate aminotransferase (AST) activity in mice fed a standard chow diet (WT/Chow), mice fed a MCD diet for 2 weeks (WT/MCD 2 w), 2-week vehicle treated mice fed a MCD diet for a total of 4 weeks (WT/MCD 4 w/V-2 w), 2-week Compound A treated mice fed a MCD diet for a total of 4 weeks (WT/MCD4 w/Compound A-2 w), 4-week vehicle treated mice fed a MCD diet for a total of 6 weeks (WT/MCD 6 w/V-4 w), and 4-week Compound A treated mice fed a MCD diet for a total of 6 weeks (WT/MCD6 w/Compound A-4 w). The MCD diet increased the levels of ALT and AST. Compound A treatment significantly reduced the level of ALT and AST compared to vehicle treated mice, thereby showing that Compound A improved liver function. (*P<0.01, WT/MCD 4 w/V-2 w vs. WT/MCD4 w/Compound A-2 w; *P<0.01, WT/MCD 6 w/V-4 w vs. WT/MCD6 w/Compound A-4 w).

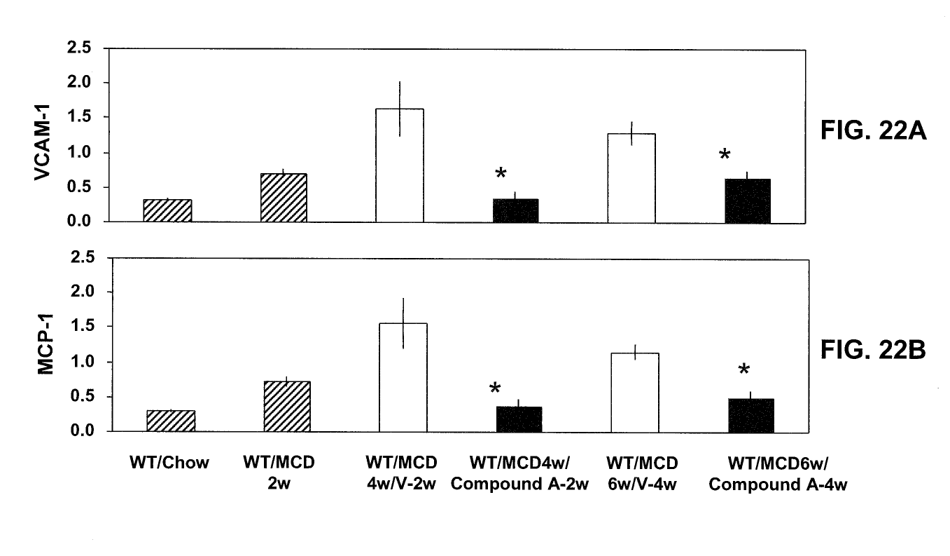

FIG. 22A shows the gene expression level of VCAM-1 and FIG. 22B shows the gene expression level of MCP-1 in the livers of WT mice fed a standard chow diet (WT/Chow), mice fed a MCD diet for 2 weeks (WT/MCD 2 w), 2-week vehicle treated mice fed a MCD diet for a total of 4 weeks (WT/MCD 4 w/V-2 w), 2-week Compound A treated mice fed a MCD diet for a total of 4 weeks (WT/MCD4 w/Compound A-2 w), 4-week vehicle treated mice fed a MCD diet for a total of 6 weeks (WT/MCD 6 w/V-4 w), and 4-week Compound A treated mice fed a MCD diet for a total of 6 weeks (WT/MCD6 w/Compound A-4 w). Compound A treatment significantly lowered the levels of the liver inflammation markers, VCAM-1 and MCP-1. (*P<0.01, WT/MCD 4 w/V-2 w vs. WT/MCD4 w/Compound A-2 w; *P<0.01, WT/MCD 6 w/V-4 w vs. WT/MCD6 w/Compound A-4 w).

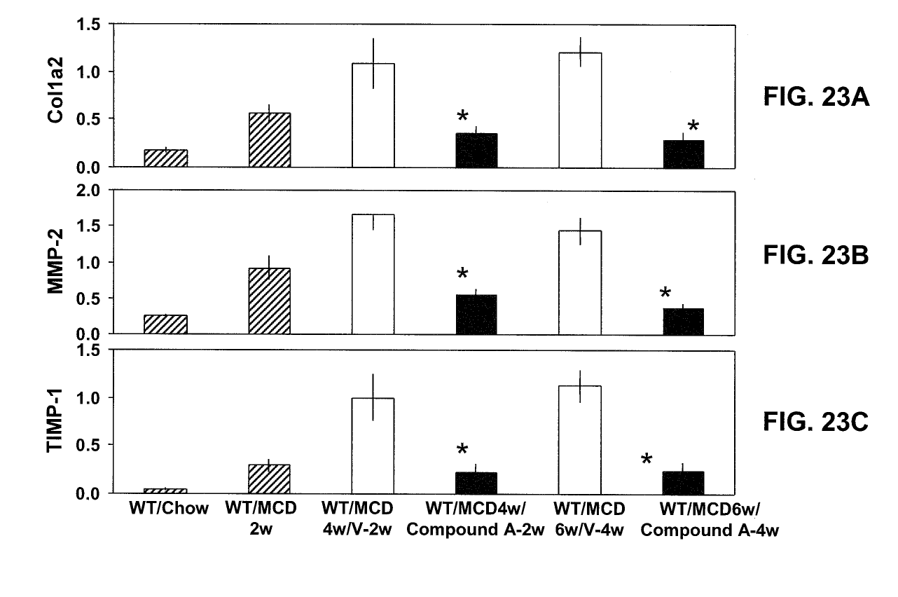

FIG. 23A shows the gene expression level of Col1a2, FIG. 23B shows the gene expression level of MMP-2, and FIG. 23C shows the gene expression level of TIMP-1 in the livers of WT mice fed a standard chow diet (WT/Chow), mice fed a MCD diet for 2 weeks (WT/MCD 2 w), 2-week vehicle treated mice fed a MCD diet for a total of 4 weeks (WT/MCD 4 w/V-2 w), 2-week Compound A treated mice fed a MCD diet for a total of 4 weeks (WT/MCD4 w/Compound A-2 w), 4-week vehicle treated mice fed a MCD diet for a total of 6 weeks (WT/MCD 6 w/V-4 w), and 4-week Compound A treated mice fed a MCD diet for a total of 6 weeks (WT/MCD6 w/Compound A-4 w). The MCD diet increased the levels of Col1a2, MMP-2, and TIMP-1. Compound A treatment significantly lowered the levels of the liver fibrosis markers, Col1a2, MMP-2, and TIMP-1 in mice fed a MCD diet. (*P<0.001, WT/MCD 4 w/V-2 w vs. WT/MCD4 w/Compound A-2 w; *P<0.01, WT/MCD 6 w/V-4 w vs. WT/MCD6 w/Compound A-4 w).

### Example 11

Human hepatoma cells, Hep3B secrete the acute phase protein, C-reactive protein (CRP) upon IL-6 treatment. Cells were cotreated with Compound A to determine if Compound A could decrease IL-6 induced CRP secretion.

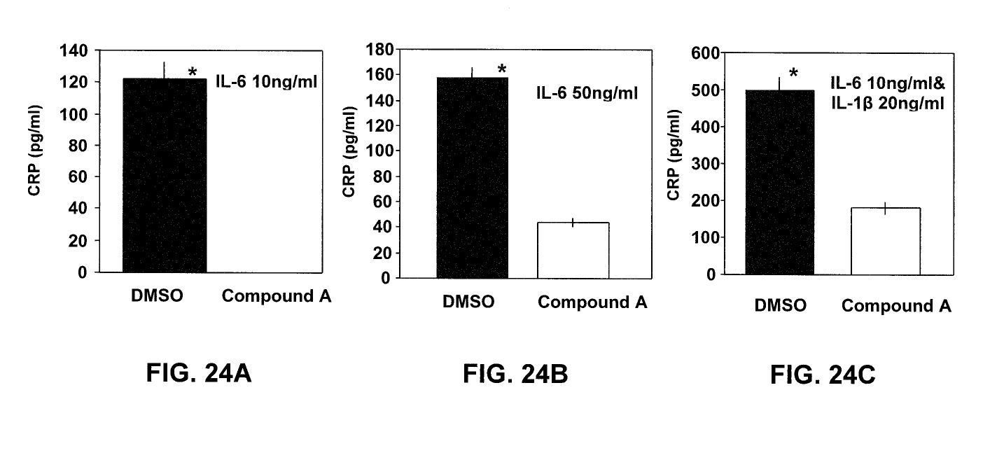

FIGS. 24A, 24B, and 24C show the levels of CRP secretion (pg/ml) in Hep3B cells after stimulation with IL-6 (10 ng/ml), or IL-6 (50 ng/ml), or IL-6 (10 ng/ml & IL-1β 20 ng/ml), and treated with vehicle (DMSO) or Compound A (5 μM). Compound A treatment inhibited IL-6 induced CRP secretion in Hep3B cells. (*P<0.001, Hep3B/DMSO vs. Hep3B/Compound A).

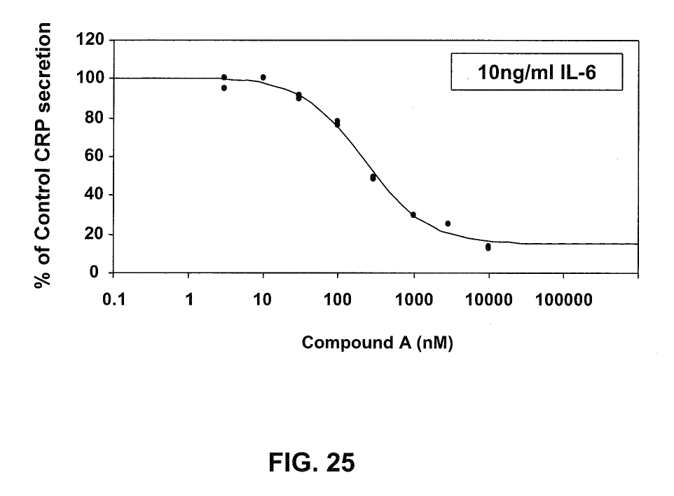

FIG. 25 shows the results of an experiment to determine the IC₅₀ of Compound A's inhibitory effect on CRP secretion in Hep3B cells treated with 10 ng/ml of IL-6. The IC₅₀ value measured when 10 ng/ml of IL-6 was used to treat the cells was 224 nM and the IC₅₀ value was 220 nM upon 50 ng/ml IL-6 treatment.

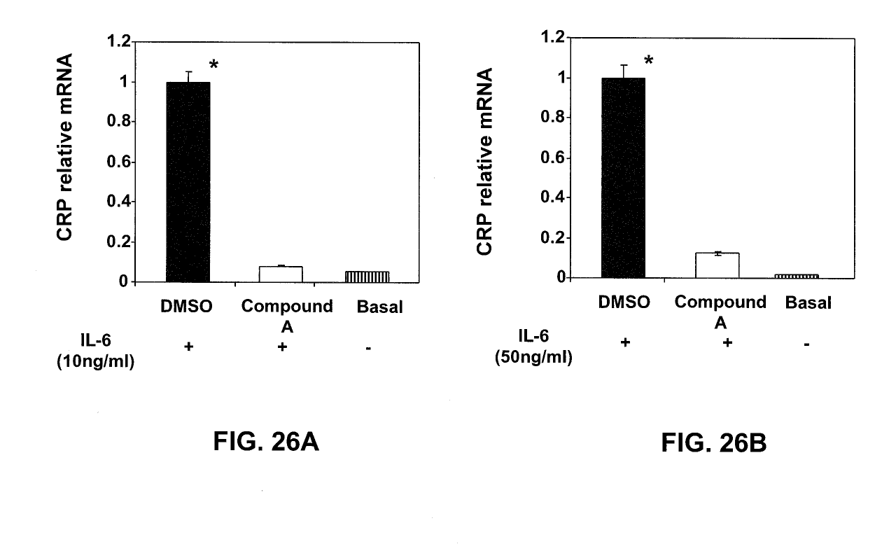

FIGS. 26A and 26B show the CRP gene expression level in Hep3B cells stimulated with IL-6 (10 ng/ml or 50 ng/ml), and treated with vehicle (DMSO) or Compound A (1 μM). These figures show that Compound A treatment reduced IL-6 induced CRP mRNA levels. (*P<0.001, Hep3B/DMSO vs. Hep3B/Compound A).

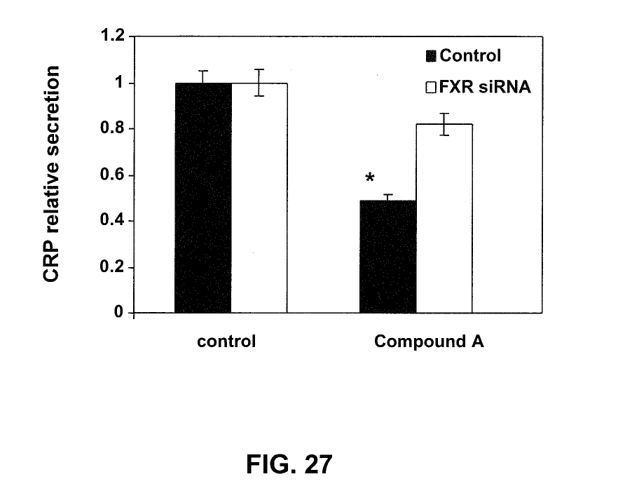

FXR siRNA studies were performed in Hep3B cells. FIG. 27 shows that FXR siRNA blocked Compound A's inhibitory effect on CRP secretion in Hep3B cells. The cells were transfected with FXR siRNA or control siRNA, stimulated with 50 ng/ml IL-6, and treated with control (DMSO) or Compound A (1 μM). CRP concentrations in the conditional media were measured by ELISA. (*P<0.01, Control siRNA/DMSO vs. Control siRNA/Compound A).

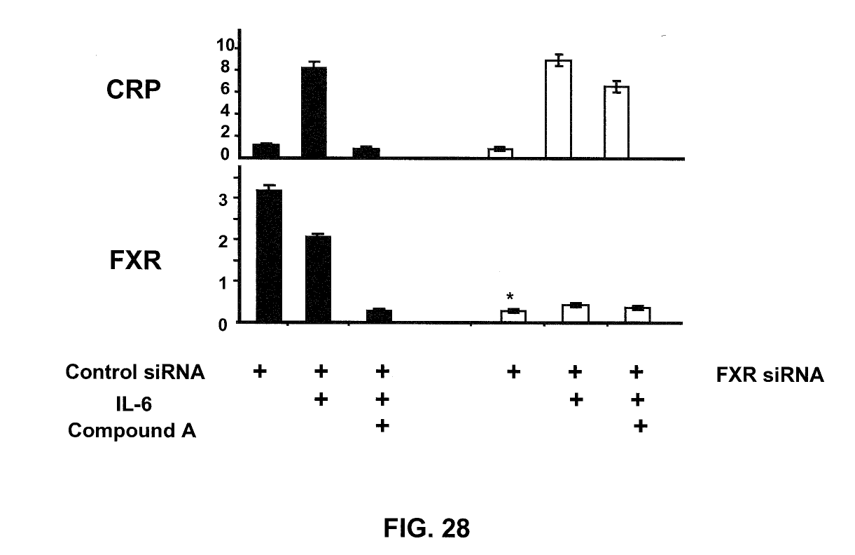

FXR siRNA also suppressed the effect of Compound A on CRP inhibition at the mRNA level. FIG. 28 shows the CRP and FXR relative gene expression levels in Hep3B cells. The Hep3B cells were transfected with FXR siRNA or control siRNA, stimulated with 50 ng/ml IL-6, and treated with control (DMSO) or Compound A (1 μM). The figure also shows that FXR mRNA was reduced to 15% of basal levels. (*P<0.001, Control siRNA vs. FXR siRNA).

### Example 12

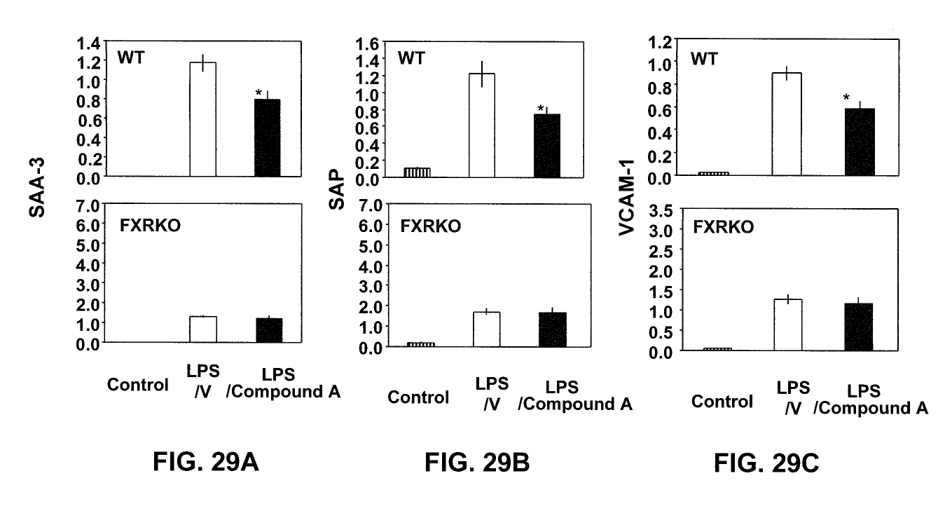

Wildtype and FXR deficient mice were treated with Compound A (30 mg/kg) or vehicle for 4 days. On day 4, mice were challenged with 2.5 micrograms of lipopolysaccharide (LPS) to induce an acute phase response. After 4 hours, the mice were euthanized and their livers were harvested for analysis of expression of various factors. FIG. 29A shows the gene expression level of serum amyloid A-3 (SAA-3), FIG. 29B shows the serum amyloid P (SAP) gene expression level, and FIG. 29C shows the VCAM-1 gene expression level in the livers of WT and FXRKO mice, vehicle treated WT and FXRKO mice challenged with LPS, and Compound A treated WT and FXRKO mice challenged with LPS. Compound A attenuated the LPS-induced murine liver acute phase response. Compound A treatment reduced SAA-3 levels by 35% in wildtype mice compared to vehicle treated wildtype mice. Compound A treatment reduced SAP levels by 40% in wildtype mice compared to vehicle treated wildtype mice. Compound A treatment reduced VCAM-1 levels by 40% in wildtype mice compared to vehicle treated wildtype mice. The data show that the effects of Compound A on SAA-3, SAP, and VCAM-1 levels are mediated by FXR because the levels of SAA-3, SAP, and VCAM-1 in LPS treated FXRKO mice were not significantly altered by Compound A treatment. (*P<0.01, WT/LPS/V vs. WT/LPS/Compound A).

### Example 13

The effects of Compound A in chemically induced murine liver injury and fibrosis were studied. Wildtype mice were treated with 30 mg/kg Compound A or vehicle for 4 days. On day 3, mice were challenged with 30 μl/kg of carbon tetrachloride (CCl4) to induce acute liver injury. After 24 hours, mice were euthanized and their livers were harvested for analysis.

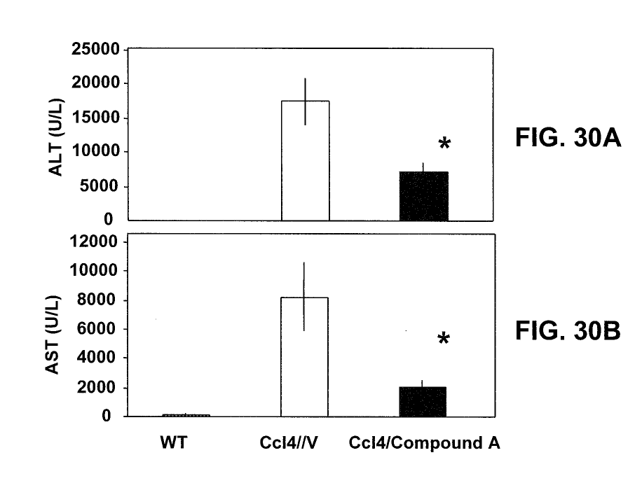

FIG. 30A shows the serum level of ALT activity and FIG. 30B shows the serum level of AST activity in WT mice, vehicle treated WT mice challenged with CCl4, and Compound A treated WT mice challenged with CCl4. Compound A treatment decreased ALT levels by 60% and AST levels by 75% compared to the vehicle treated mice and had a liver protection effect. (*P<0.01, WT/CCl4/V vs. WT/CCl4/Compound A).

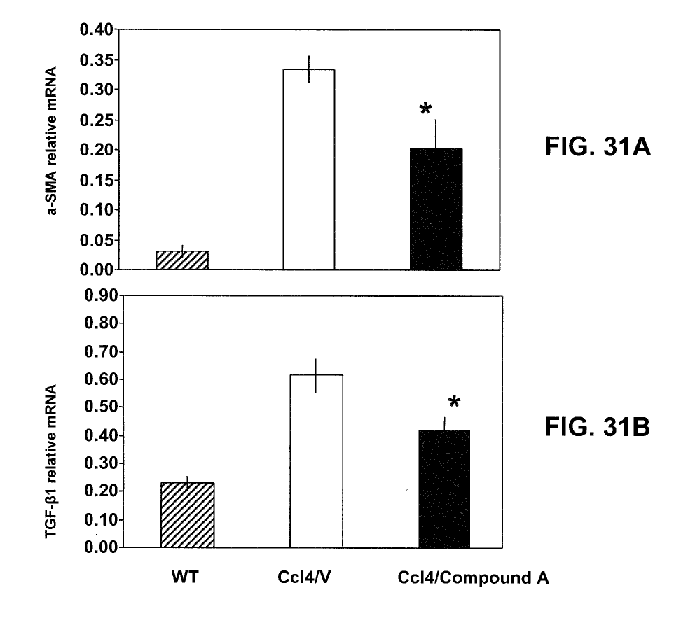

FIG. 31A shows the gene expression level of α smooth muscle actin (a-SMA) mRNA and FIG. 31B shows the gene expression level of transforming growth factor β1 (TGF-β1) mRNA in the livers of the WT mice, vehicle treated WT mice challenged with CCl4, and Compound A treated WT mice challenged with CCl4. Expression was normalized to GAPDH mRNA. Compound A treatment decreased a-SMA levels by 40% and TGF-β levels by 30% compared to the vehicle treated mice and protected the liver from fibrosis. (*P<0.01, WT/CCl4/V vs. WT/CCl4/Compound A).

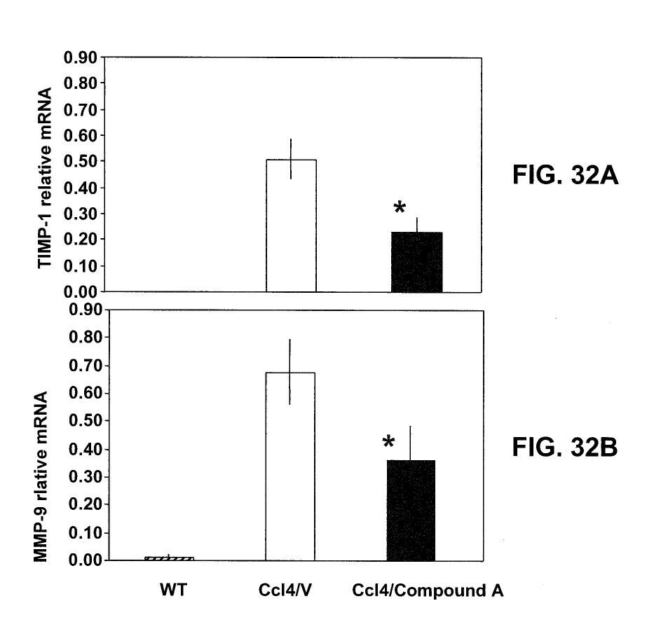

FIG. 32A shows the gene expression level of TIMP-1 mRNA and FIG. 32B shows the gene expression level of MMP-9 mRNA in the livers of WT mice, vehicle treated WT mice challenged with CCl4, and Compound A treated WT mice challenged with CCl4. Expression was normalized to GAPDH mRNA. Compound A treatment decreased TIMP-1 levels by 60% and MMP-9 levels by 75% compared to the vehicle treated mice and had a liver protection effect. (*P<0.04, WT/CCl4/V vs. WT/CCl4/Compound A).

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method of treating nonalcoholic fatty liver disease (NAFLD) in a patient, the method comprising administering to the patient a therapeutically effective amount of at least one farnesoid X receptor (FXR) agonist.

2. The method of claim 1, wherein the nonalcoholic fatty liver disease is characterized by at least one of steatosis, nonalcoholic steatohepatitis (NASH), NAFLD induced hepatitis, NAFLD induced fibrosis, and NAFLD induced cirrhosis.

3. The method of claim 1, wherein the at least one FXR agonist reduces at least one feature of nonalcoholic fatty liver disease selected from neutral lipid deposition, intracellular lipid droplet formation, Kupffer cell activation, inflammatory cell infiltration, inflammatory cholangitis, portal inflammation, fibrosis, oxidative stress, and increased serum C-reactive protein (CRP) level.

4. The method of claim 1, wherein administration of the at least one FXR agonist to the patient causes at least one of a reduction in the level of at least one of vascular cell adhesion molecule-1 (VCAM-1), intercellular adhesion molecule-1 (ICAM-1), tumor necrosis factor α (TNFα), monocyte chemotactic protein-1 (MCP-1), keratinocyte-derived chemokine (KC), collagen, type 1, alpha 2 (Col1a2), transforming growth factor β (TGF-β), α smooth muscle actin (a-SMA), at least one matrix metalloproteinase (MMP), at least one positive acute phase protein, and Cytochrome P450 2E1 (CYP2 μl), and a modulation in the level of tissue inhibitor of metalloproteinase 1 (TIMP-1) in the patient.

5. The method of claim 1, wherein the at least one FXR agonist reduces the serum level of at least one of alanine aminotransferase (ALT), aspartate aminotransferase (AST), and cytokeratin 18 (CK-18) in the patient.

6. The method of claim 1, wherein the at least one FXR agonist elevates the level of at least one FXR target in the patient selected from fatty acid synthase (FAS), small heterodimer partner (SHP), bile salt export pump (BSEP), and multiple drug resistance-2 (MDR2).

7. The method of claim 1, wherein the at least one FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl) 1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-d]azepine-8-carboxylic acid ethyl ester;
6-(4-fluoro-benzoyl)-3,6,7,8-tetrahydro-imidazo[4,5-d]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3-(4-fluorobenzoyl)-2-methyl-1,2,3,6-tetrahydroazepino[4,5-b]indole-5-carboxylate; and
n-propyl 3-(4-fluorobenzoyl)-2-methyl-8-fluoro-1,2,3,6-tetrahydroazepino[4,5-b]indole-5-carboxylate.

8.-16. (canceled)

17. A method of treating a patient with existing cholesterol gallstone disease, the method comprising administering to the patient a therapeutically effective amount of at least one FXR agonist.

18. The method of claim 17, wherein the patient is characterized by at least one feature selected from is highly symptomatic, is awaiting a cholecystectomy, and is not a suitable candidate for surgical intervention.

19. The method of claim 17, wherein the at least one FXR agonist reduces at least one feature of cholesterol gallstone disease selected from gallstone incidence, gallstone dissolution time, bile cholesterol levels, bile salt/phospholipid ratios, biliary symptoms, and gallbladder inflammation in the patient.

20. The method of claim 17, wherein the at least one FXR agonist reduces at least one feature selected from neutral lipid deposition, intracellular lipid droplet formation, Kupffer cell activation, inflammatory cell infiltration, inflammatory cholangitis, portal inflammation, fibrosis, oxidative stress, and acute phase response in the liver of the patient.

21. The method of claim 17, wherein administration of the at least one FXR agonist to the patient causes at least one of a reduction in the level of at least one of VCAM-1, ICAM-1, TNFα, MCP-1, KC, Col1a2, TGF-β, a-SMA, at least one MMP, at least one positive acute phase protein, and CYP2E1 and a modulation in the level of TIMP-1 in the patient.

22. The method of claim 17, wherein the at least one FXR agonist reduces the serum level of at least one of ALT, AST, and CK-18 in the patient.

23. The method of claim 17, wherein the at least one FXR agonist elevates the level of at least one FXR target in the patient selected from FAS, SHP, BSEP, and MDR2.

24. The method of claim 17, wherein the at least one FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl)1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro-4H-thieno[2,3-d]azepine-8-carboxylic acid ethyl ester;
6-(4-fluoro-benzoyl)-3,6,7,8-tetrahydro-imidazo[4,5-d]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate;
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3-(4-fluorobenzoyl)-2-methyl-1,2,3,6-tetrahydroazepino[4,5-b]indole-5-carboxylate; and
n-propyl 3-(4-fluorobenzoyl)-2-methyl-8-fluoro-1,2,3,6-tetrahydroazepino[4,5-b]indole-5-carboxylate.

25. (canceled)

26. The method of claim 17, wherein the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress, and acute phase response in the liver, and an elevated level of at least one of VCAM-1, ICAM-1, TNFα, MCP-1, KC, TIMP-1, Col1a2, TGF-β, a-SMA, at least one MMP, at least one positive acute phase protein, CYP2E1, ALT, AST, and CK-18.

27. (canceled)

28. The method of claim 26, wherein the at least one FXR agonist reduces at least one feature of cholesterol gallstone disease selected from gallstone incidence, gallstone dissolution time, bile cholesterol levels, bile salt/phospholipid ratios, biliary symptoms, and gallbladder inflammation.

29. The method of claim 26, wherein the at least one FXR agonist reduces at least one feature of cholesterol gallstone disease selected from neutral lipid deposition, intracellular lipid droplet formation, inflammatory cell infiltration, inflammatory cholangitis, portal inflammation, fibrosis, oxidative stress, and acute phase response in the liver of the patient.

30. (canceled)

31. The method of claim 26, wherein the at least one FXR agonist reduces the serum level of at least one of AST, ALT, and CK-18 in the patient.

32. (canceled)

33. The method of claim 26, wherein the at least one FXR agonist is selected from:
(3,4-difluoro-benzoyl)-4,4-dimethyl-5,6-dihydro-4H-thieno[2,3-d]azepine-8-carboxylic acid ethyl ester;
3-(3,4-difluorobenzoyl)-1,1,6-trimethyl-1,2,3,6-tetrahydroazepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid ethyl ester;
3-(3,4-difluoro-benzoyl)-1,1-dimethylene-1,2,3,6-tetrahydro-azepino[4,5-b]indole-5-carboxylic acid isopropyl ester;
3-(3,4-difluorobenzoyl)-1,1-tetramethylene-1,2,3,6-tetrahydroazepino[4,5-b]indole-5-carboxylic acid ethyl ester;
3-(3,4-difluoro-benzoyl)-1,1-trimethylene-1,2,3,6-tetrahydro-azepino[4,5-b]indole-5-carboxylic acid ethyl ester;
3-(3,4-difluorobenzoyl)-1-methyl-1,2,3,6-tetrahydroazepino[4,5-b]indole-5-carboxylic acid cyclobutylamide;
3-(3,4-difluorobenzoyl)-2-methyl-1,2,3,6-tetrahydroazepino[4,5-b]indole-5-carboxylic acid cyclobutylamide;
3-(3-fluorobenzoyl)-1,2,3,6-tetrahydroazepino[4,5-b]indole-5-carboxylic acid ethyl ester;
3-(4-fluoro-benzoyl)-1,1-dimethyl-1,2,3,4,5,6,7,8,9,10-decahydroazepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,1-dimethyl-1,2,3,6-tetrahydro-azepino[4,5-b]indole-5-carboxylic acid isopropylamide;
3-(4-fluoro-benzoyl)-1,1-dimethyl-9-(3-methyl-butyrylamino)-1,2,3,6-tetrahydro-azepino[4,5-b]indole-5-carboxylic acid ethyl ester;
3-(4-fluoro-benzoyl)-1,1-dimethyl-9-phenylacetylamino-1,2,3,6-tetrahydro-azepino[4,5-b]indole-5-carboxylic acid ethyl ester;
3-(4-fluoro-benzoyl)-1,1-dimethyl-1,2,3,6,7,8,9,10-octahydro-azepino[4,5-b]indole-5-carboxylic acid ethyl ester;
3-(4-fluoro-benzoyl)-1,2,3,4,5,6,7,8,9,10-decahydro-azepino[4,5-b]indole-5-carboxylic acid ethyl ester;
3-(4-fluoro-benzoyl)1,2,3,6,7,8,9,10-octahydro-azepino[4,5-b]indole-5-carboxylic acid ethyl ester;
3-(4-fluorobenzoyl)-1,2,3,6-tetrahydroazepino[4,5-b]indole-5-carboxylic acid cyclobutylamide;
3-(4-fluorobenzoyl)-2-methyl-1,2,3,6-tetrahydroazepino[4,5-b]indole-5-carboxylic acid cyclobutylamide;
6-(3,4-difluoro-benzoyl)-1,4,4-trimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid 2-ethyl ester 8-isopropyl ester;
6-(3,4-difluoro-benzoyl)-4,4-dimethyl-1,4,5,6-tetrahydro-pyrrolo[2,3-d]azepine-2,8-dicarboxylic acid dimethyl ester;
6-(3,4-difluoro-benzoyl)-4,4-dimethyl-1,4,5,6-tetrahydro-pyrrolo[2,3-d]azepine-2,8-dicarboxylic acid diethyl ester;
6-(3,4-difluoro-benzoyl)-4,4-dimethyl-5,6-dihydro-4H-thieno[2,3-d]azepine-8-carboxylic acid ethyl ester;
6-(3,4-difluoro-benzoyl)-5,6-dihydro4H-thieno[2,3-d]azepine-8-carboxylic acid ethyl ester;
6-(4-fluoro-benzoyl)-3,6,7,8-tetrahydro-imidazo[4,5-d]azepine-4-carboxylic acid ethyl ester;
9-(1-benzyl-3,3-dimethyl-ureido)-3-(4-fluoro-benzoyl)-1,1-dimethyl-1,2,3,6-tetrahydro-azepino[4,5-b]indole-5-carboxylic acid ethyl ester;
9-(2,2-dimethyl-propionylamino)-3-(4-fluoro-benzoyl)-1,1-dimethyl-1,2,3,6-tetrahydro-azepino[4,5-b]indole-5-carboxylic acid ethyl ester;
9-(acetyl-methyl-amino)-3-(4-fluoro-benzoyl)-1,1-dimethyl-1,2,3,6-tetrahydro-azepino[4,5-b]indole-5-carboxylic acid ethyl ester;
9-[benzyl-(2-thiophen-2-yl-acetyl)-amino]-3-(4-fluoro-benzoyl)-1,1-dimethyl-1,2,3,6-tetrahydro-azepino[4,5-b]indole-5-carboxylic acid ethyl ester;
9-dimethylamino-3-(4-fluorobenzoyl)-1,1-dimethyl-1,2,3,6-tetrahydroazepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid ethyl ester;
9-fluoro-3-(3,4-difluoro-benzoyl)-1,1-dimethyl-1,2,3,6-tetrahydro-azepino[4,5-b]indole-5-carboxylic acid isopropylamide;
9-fluoro-3-(4-fluoro-benzoyl)-1,1-dimethyl-1,2,3,6-tetrahydro-azepino[4,5-b]indole-5-carboxylic acid ethyl ester;
9-fluoro-3-(4-fluoro-benzoyl)-1,1-dimethyl-1,2,3,6-tetrahydro-azepino[4,5-b]indole-5-carboxylic acid isopropyl ester;
9-fluoro-3-cyclohexanecarbonyl-1,1-dimethyl-1,2,3,6-tetrahydro-azepino[4,5-b]indole-5-carboxylic acid ethyl ester;
cyclobutyl 3-(3,4-difluorobenzoyl)-1,1-dimethyl-1,2,3,6-tetrahydroazepino[4,5-b]indole-5-carboxamide;
diethyl 3-(4-fluorobenzoyl-1,2,3,6-tetrahydroazepino[4,5-b]indole-2,5-dicarboxylate;
ethyl 1,1-dimethyl-1,2,3,6-tetrahydro-azepino[4,5-b]indole5-carboxylate;
ethyl 1,1-dimethyl-3-(4-fluorobenzoyl)-1,2,3,6-tetrahydro-azepino[4,5-b]indole-5-carboxylate;
ethyl 3-(3,4-difluorobenzoyl)-1,1-dimethyl-1,2,3,6-tetrahydroazepino[4,5-b]indole-5-carboxylate;
ethyl 3-(3,4-difluorobenzoyl)-1-methyl-1,2,3,6-tetrahydroazepino[4,5-b]indole-5-carboxylate;
ethyl 3-(4-chlorobenzoyl)-1,1-dimethyl-1,2,3,6-tetrahydroazepino[4,5-b]indole-5-carboxylate
ethyl 3-(4-chlorobenzoyl)-1-methyl-1,2,3,6-tetrahydroazepino[4,5-b]indole-5-carboxylate;
ethyl 3-(4-fluorobenzoyl)-1,2,3,6-tetrahydroazepino[4,5-b]indole-5-carboxylate;
ethyl 3-(4-fluorobenzoyl)-1-methyl-1,2,3,6-tetrahydro-azepino[4,5-b]indole-5-carboxylate;
isopropyl 3-(3,4-difluorobenzoyl)-1,1-dimethyl-1,2,3,6-tetrahydroazepino[4,5-b]indole-5-carboxylate;
isopropyl 3-(3,4-difluorobenzoyl)-1-methyl-1,2,3,6-tetrahydroazepino[4,5-b]indole-5-carboxylate;
n-propyl 3-(4-fluorobenzoyl)-2-methyl-1,2,3,6-tetrahydroazepino[4,5-b]indole-5-carboxylate; and
n-propyl 3-(4-fluorobenzoyl)-2-methyl-8-fluoro-1,2,3,6-tetrahydroazepino[4,5-b]indole-5-carboxylate.

34.-44. (canceled)
